SEQUENCE LISTING (28 sequences)

SEQ ID NO: 1 - protein, 120 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 2 - protein, 111 aa, Artificial Sequence
  Synthetic Construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 3 - protein, 133 aa, Artificial Sequence
  Synthetic Construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 4 - protein, 121 aa, Artificial Sequence
  Synthetic Construct
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSS

SEQ ID NO: 5 - protein, 107 aa, Artificial Sequence
  Synthetic Construct
DIQMTQSPSSLSASVGDRVTITCRASGNIYNYLAWYQQKPGKVPKLLIYDAKTLADGVPSRFSGSGSGTDFTLTISSLQPEDVATYYCQHFWSLPFTFGQGTKLEIK

SEQ ID NO: 6 - protein, 125 aa, Artificial Sequence
  Synthetic Construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSS

SEQ ID NO: 7 - protein, 109 aa, Artificial Sequence
  Synthetic Construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 8 - protein, 597 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 9 - protein, 448 aa, Artificial Sequence
  Synthetic Construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 10 - protein, 218 aa, Artificial Sequence
  Synthetic Construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 607 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ

SEQ ID NO: 12 - protein, 444 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 13 - protein, 218 aa, Artificial Sequence
  Synthetic Construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 674 aa, Artificial Sequence
  Synthetic Construct
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 15 - protein, 449 aa, Artificial Sequence
  Synthetic Construct
QVQLVQSGAEVKKPGASVKVSCKASGFNIKDYFLHWVRQAPGQGLEWMGWINPDNGNTVYAQKFQGRVTMTADTSTSTVYMELSSLRSEDTAVYYCTRRDYTYEKAALDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 16 - protein, 214 aa, Artificial Sequence
  Synthetic Construct
DIQMTQSPSSLSASVGDRVTITCRASGNIYNYLAWYQQKPGKVPKLLIYDAKTLADGVPSRFSGSGSGTDFTLTISSLQPEDVATYYCQHFWSLPFTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 232 aa, Artificial Sequence
  Synthetic Construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 679 aa, Artificial Sequence
  Synthetic Construct
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASDAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSEVQLQQSGAELVRPGALVKLSCKTSGFNIKDYFLHWVRQRPDQGLEWIGWINPDNGNTVYDPKFQGTASLTADTSSNTVYLQLSGLTSEDTAVYFCTRRDYTYEKAALDYWGQGASVIVFSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPGK

SEQ ID NO: 19 - protein, 446 aa, Artificial Sequence
  Synthetic Construct
EVQLQQSGAELVRPGALVKLSCKTSGFNIKDYFLHWVRQRPDQGLEWIGWINPDNGNTVYDPKFQGTASLTADTSSNTVYLQLSGLTSEDTAVYFCTRRDYTYEKAALDYWGQGASVIVFSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGK

SEQ ID NO: 20 - protein, 214 aa, Artificial Sequence
  Synthetic Construct
AIQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 21 - protein, 211 aa, Artificial Sequence
  Synthetic Construct
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDC

SEQ ID NO: 22 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 23 - protein, 513 aa, Homo sapiens
QFPRQCATVEALRSGMCCPDLSPVSGPGTDRCGSSSGRGRCEAVTADSRPHSPQYPHDGRDDREVWPLRFFNRTCHCNGNFSGHNCGTCRPGWRGAACDQRVLIVRRNLLDLSKEEKNHFVRALDMAKRTTHPLFVIATRRSEEILGPDGNTPQFENISIYNYFVWTHYYSVKKTFLGVGQESFGEVDFSHEGPAFLTWHRYHLLRLEKDMQEMLQEPSFSLPYWNFATGKNVCDICTDDLMGSRSNFDSTLISPNSVFSQWRVVCDSLEDYDTLGTLCNSTEDGPIRRNPAGNVARPMVQRLPEPQDVAQCLEVGLFDTPPFYSNSTNSFRNTVEGYSDPTGKYDPAVRSLHNLAHLFLNGTGGQTHLSPNDPIFVLLHTFTDAVFDEWLRRYNADISTFPLENAPIGHNRQYNMVPFWPPVTNTEMFVTAPDNLGYTYEIQWPSREFSVPEIIAIAVVGALLLVALIFGTASYLIRARRSMDEANQPLLTDQYQCYAEEYEKLQNPNQSVV

SEQ ID NO: 24 - protein, 603 aa, Artificial Sequence
  Synthetic Construct
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDAPEVQFSWFVDDVEVHTAQTQPREEQFNSTFRSVSELPIMHQDWLNGKEFKCRVNSAAFGAPIEKTISKTKGRPKAPQVYTIPPCKEQMAKDKVSLWCMITDFFPEDITVEWQWNGQPAENYKNTQPIMDTDGSYFVYSKLNVQKSNWEAGNTFTCSVLHEGLHNHHTEKSLSHSPGGGGGSGGGGSGGGGSAPASSSTSSSTAEAQQQQQQQQQQQQHLEQLLMDLQELLSRMENYRNLKLPRMLTAKFALPKQATELKDLQCLEDELGPLRHVLDGTQSKSFQLEDAENFISNIRVTVVKLKGSDNTFECQFDDESATVVDFLRRWIAFAQSIISTSPQ

SEQ ID NO: 25 - protein, 440 aa, Artificial Sequence
  Synthetic Construct
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDAPEVQFSWFVDDVEVHTAQTQPREEQFNSTFRSVSELPIMHQDWLNGKEFKCRVNSAAFGAPIEKTISKTKGRPKAPQVCTIPPPKEQMAKDKVSLSCAITDFFPEDITVEWQWNGQPAENYKNTQPIMDTDGSYFVVSKLNVQKSNWEAGNTFTCSVLHEGLHNHHTEKSLSHSPGK

SEQ ID NO: 26 - protein, 215 aa, Artificial Sequence
  Synthetic Construct
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 27 - protein, 450 aa, Artificial Sequence
  Synthetic Construct
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 28 - protein, 218 aa, Artificial Sequence; identical to SEQ ID NO: 13
  Synthetic Construct

(19) United States
(12) Patent Application Publication
(10) Pub. No.: US 2024/0262913 A1
(43) Pub. Date: Aug. 8, 2024

(54) COMBINATION THERAPY OF PD-1-TARGETED IL-2 VARIANT IMMUNOCONJUGATE AND ANTI-TYRP1/ANTI-CD3 BISPECIFIC ANTIBODIES

(71) Applicant: Hoffmann-La Roche Inc., Little Falls, NJ (US)

(72) Inventors: Laura CODARRI DEAK, Au (CH); Christian KLEIN, Weilheim (DE); Valeria NICOLINI, Erlenbach (CH); Pablo UMAÑA, Wollerau (CH)

(73) Assignee: Hoffmann-La Roche Inc., Little Falls, NJ (US)

(21) Appl. No.: 18/549,877

(22) PCT Filed: Mar. 8, 2022
(86) PCT No.: PCT/EP2022/055796

§ 371 Date: Sep. 8, 2023

(30) Foreign Application Priority Data
Mar. 9, 2021 (EP) 21161465.6

(51) Int. Cl. C07K 16/28; A61K 39/00; A61P 35/04; C07K 14/55; C07K 16/40

(52) U.S. Cl. CPC C07K 16/2818; A61P 35/04; C07K 14/55; C07K 16/2809; C07K 16/40; A61K 2039/507; C07K 2317/31; C07K 2317/71; C07K 2317/73; C07K 2319/00

(57) ABSTRACT

The present invention relates to the combination therapy of specific PD-1-targeted IL-2 variant immunoconjugate with specific antibodies which bind human TYRP1 and CD3.

### FIELD OF THE INVENTION

The present invention relates to the combination therapy of PD-1-targeted IL-2 variant immunoconjugates with bispecific antibodies which bind to human TYRP1 and CD3.

### BACKGROUND OF THE INVENTION

Cancer is the leading cause of death in economically developed countries and the second leading cause of death in developing countries. Despite recent advances in chemotherapy and the development of agents targeted at the molecular level to interfere with the transduction and regulation of growth signals in cancer cells, the prognosis of patients with advanced cancer remains poor in general. Consequently, there is a persisting and urgent medical need to develop new therapies that can be added to existing treatments to increase survival without causing unacceptable toxicity.

Interleukin 2 (IL-2) is a cytokine that activates lymphocytes and natural killer (NK) cells. IL-2 has been shown to have anti-tumor activity; however, high levels of IL-2 lead to pulmonary toxicity, and the anti-tumor activity of IL-2 is limited by a number of inhibitory feedback loops.

Based on its anti-tumor efficacy, high-dose IL-2 (aldesleukin, marketed as Proleukin®) treatment has been approved for use in patients with metastatic renal cell carcinoma (RCC) and malignant melanoma in the US, and for patients with metastatic RCC in the European Union. However, as a consequence of the mode of action of IL-2, the systemic and untargeted application of IL-2 may considerably compromise anti-tumor immunity via induction of T_{reg} cells and AICD. An additional concern of systemic IL-2 treatment is related to severe side-effects upon intravenous administration, which include severe cardiovascular, pulmonary edema, hepatic, gastrointestinal (GI), neurological, and hematological events (Proleukin (aldesleukin) Summary of Product Characteristics [SmPC]: http://www.medicines.org.uk/emc/medicine/19322/SPC/ (accessed May 27, 2013)). Low-dose IL-2 regimens have been tested in patients, although at the expense of suboptimal therapeutic results. Taken together, therapeutic approaches utilizing IL-2 may be useful for cancer therapy if the liabilities associated with its application can be overcome. Immunoconjugates comprising a PD-1-targeted antigen binding moiety and an IL-2-based effector moiety are described in e.g. WO 2018/184964 A1.

Programmed cell death protein 1 (PD-1 or CD279) is an inhibitory member of the CD28 family of receptors, that also includes CD28, CTLA-4, ICOS and BTLA. PD-1 is a cell surface receptor and is expressed on activated B cells, T cells, and myeloid cells (Okazaki et al (2002) Curr. Opin. Immunol. 14: 391779-82; Bennett et al. (2003) J Immunol 170:711-8). The structure of PD-1 is a monomeric type 1 transmembrane protein, consisting of one immunoglobulin variable-like extracellular domain and a cytoplasmic domain containing an immunoreceptor tyrosine-based inhibitory motif (ITIM) and an immunoreceptor tyrosine-based switch motif (ITSM). Two ligands for PD-1 have been identified, PD-L1 and PD-L2, that have been shown to downregulate T cell activation upon binding to PD-1 (Freeman et al (2000) J Exp Med 192: 1027-34; Latchman et al (2001) Nat Immunol 2:261-8; Carter et al (2002) Eur J Immunol 32:634-43). Both PD-L1 and PD-L2 are B7 homologs that bind to PD-1, but do not bind to other CD28 family members. One ligand for PD-1, PD-L1 is abundant in a variety of human cancers (Dong et al (2002) Nat. Med 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat 7. Acad. ScL USA 99: 12293-7; Brown et al. (2003) J. Immunol. 170:1257-66). Antibodies that bind to PD-1 are described in e.g. WO 2017/055443 A1.

CD3 (cluster of differentiation 3) is a protein complex composed of four subunits, the CD3γ chain, the CD3δ chain, and two CD3& chains. CD3 associates with the T-cell receptor and the Z chain to generate an activation signal in T lymphocytes.

CD3 has been extensively explored as drug target. Monoclonal antibodies targeting CD3 have been used as immunosuppressant therapies in autoimmune diseases such as type I diabetes, or in the treatment of transplant rejection. The CD3 antibody muromonab-CD3 (OKT3) was the first monoclonal antibody ever approved for clinical use in humans, in 1985.

A more recent application of CD3 antibodies is in the form of bispecific antibodies, binding CD3 on the one hand and a tumor cell antigen on the other hand (Clynes and Desjarlais (2019) Annu. Rev. Med. 70:437-50). The simultaneous binding of such an antibody to both of its targets will force a temporary interaction between target cell and T cell, causing activation of any cytotoxic T cell and subsequent lysis of the target cell. For this purpose bispecific antibodies binding CD3 and the tumor cell antigen TYRP1 have been developed; as described e.g. in WO 2020/127619 A1. TYRP1 as a target is present in melanoma cells and in melanocytes where it is involved in melanin synthesis and also affects melanocyte proliferation and survival in humans. TYRP1 antibodies have been previously described (Boross et al. (2014) Immunol Lett. 160(2):151-7) and been tested in clinical trials (Khalil et al. (2016) Clin Cancer Res. 22(21): 5204-5210). For use in mouse models, TYRP1 and T cell bispecific surrogate antibodies have been described (Benonisson et al. (2019) Mol Cancer Ther. (2):312-322; Labrijn et al. (2017) Sci Rep. 7(1):2476) where they mediated anti-tumor efficacy, but could not induce long term response/cure.

There is still a need for new compounds and combinations which have the potential to significantly contribute to the treatment of patients. Thus, we herein describe a novel combination therapy of PD1-IL2v and bispecific antibodies which bind to TYRP1 and CD3.

### SUMMARY OF THE INVENTION

The invention comprises the combination therapy of a PD-1-targeted IL-2 variant immunoconjugate with an anti-TYRP1/anti-CD3 bispecific antibody for use as a combination therapy in the treatment of cancer, for use as a combination therapy in the prevention or treatment of metastasis, for use as a combination therapy in treating or delaying progression of an immune related disease such as tumor immunity, or for use as a combination therapy in stimulating an immune response or function, such as T cell activity, wherein the PD-1-targeted IL-2 variant immunoconjugate comprises a heavy chain variable domain VH of SEQ ID NO: 1 and a light chain variable domain VL of SEQ ID NO: 2, and the polypeptide sequence of SEQ ID NO: 3, and wherein the anti-TYRP1/anti-CD3 bispecific antibody comprises a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7.

In one aspect, the PD-1-targeted IL-2 variant immunoconjugate in combination with an anti-TYRP1/anti-CD3 bispecific antibody may be for use in the treatment of breast cancer, lung cancer, colon cancer, ovarian cancer, melanoma cancer, bladder cancer, renal cancer, kidney cancer, liver cancer, head and neck cancer, colorectal cancer, melanoma, pancreatic cancer, gastric carcinoma cancer, esophageal cancer, mesothelioma, prostate cancer, leukemia, lymphomas, myelomas.

In a preferred aspect, the PD-1-targeted IL-2 variant immunoconjugate in combination with an anti-TYRP1/anti-CD3 bispecific antibody may be for use in the treatment of melanoma or cancer of melanocytic origin. In a preferred aspect, the PD-1-targeted IL-2 variant immunoconjugate in combination with an anti-TYRP1/anti-CD3 bispecific antibody may be for use in the treatment of TYRP1 expressing cancers.

In one aspect, the PD-1-targeted IL-2 variant immunoconjugate in combination with an anti-TYRP1/anti-CD3 bispecific antibody is characterized in that the antibody component of the immunoconjugate and the antibody are of human IgG₁ or human IgG₄ subclass.

In a further aspect, the PD-1-targeted IL-2 variant immunoconjugate in combination with an anti-TYRP1/anti-CD3 bispecific antibody is characterized in that the antibody components of the immunoconjugate and the bispecific antibody have reduced or minimal effector function.

In one aspect, the minimal effector function results from an effectorless Fc mutation.

In one aspect, the effectorless Fc mutation is L234A/L235A or L234A/L235A/P329G or N297A or D265A/N297A.

In a particular aspect, the invention provides a PD-1-targeted IL-2 variant immunoconjugate in combination with an anti-TYRP1/anti-CD3 bispecific antibody, wherein the PD-1-targeted IL-2 variant immunoconjugate comprises i) a polypeptide sequence of SEQ ID NO: 8 or SEQ ID NO: 9 or SEQ ID NO: 10, ii) the polypeptide sequence of SEQ ID NO: 8, and SEQ ID NO: 9 and SEQ ID NO: 10, or iii) the polypeptide sequence of SEQ ID NO: 11 and SEQ ID NO: 12 and SEQ ID NO: 13 and wherein the anti-TYRP1/anti-CD3 bispecific antibody comprises a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7.

In a more particular aspect, the invention provides a PD-1-targeted IL-2 variant immunoconjugate in combination with an anti-TYRP1/anti-CD3 bispecific antibody, wherein the PD-1-targeted IL-2 variant immunoconjugate comprises i) a polypeptide sequence of SEQ ID NO: 8 or SEQ ID NO: 9 or SEQ ID NO: 10, ii) the polypeptide sequence of SEQ ID NO: 8, and SEQ ID NO: 9 and SEQ ID NO: 10, or iii) the polypeptide sequence of SEQ ID NO: 11 and SEQ ID NO: 12 and SEQ ID NO: 13, and wherein the anti-TYRP1/anti-CD3 bispecific antibody comprises i) a polypeptide sequence of SEQ ID NO: 14 or SEQ ID NO: 15 or SEQ ID NO: 16 or SEQ ID NO: 17, ii) a polypeptide sequence of SEQ ID NO: 14 and SEQ ID NO: 15 and SEQ ID NO: 16 and SEQ ID NO: 17, or iii) a polypeptide sequence of SEQ ID NO: 18 and SEQ ID NO: 19 and SEQ ID NO: 20 and SEQ ID NO: 21.

In one aspect, the invention provides a PD-1-targeted IL-2 variant immunoconjugate in combination with an anti-TYRP1/anti-CD3 bispecific antibody for use in i) inhibition of tumor growth in a tumor; and/or ii) enhancing median and/or overall survival of subjects with a tumor; wherein PD-1 is presented on immune cells, particularly T cells, or in a tumor cell environment, wherein the PD-1 targeted IL-2 variant immunoconjugate used in the combination therapy is characterized in comprising i) a heavy chain variable domain VH of SEQ ID NO: 1 and a light chain variable domain VL of SEQ ID NO: 2, and the polypeptide sequence of SEQ ID NO: 3, ii) a polypeptide sequence of SEQ ID NO: 8 or SEQ ID NO: 9 or SEQ ID NO: 10, iii) the polypeptide sequence of SEQ ID NO: 8, and SEQ ID NO: 9 and SEQ ID NO: 10, or iv) the polypeptide sequence of SEQ ID NO: 11 and SEQ ID NO: 12 and SEQ ID NO: 13, and the anti-TYRP1/anti-CD3 bispecific antibody used in the combination therapy is characterized in comprising i) a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7; ii) a polypeptide sequence of SEQ ID NO: 14 or SEQ ID NO: 15 or SEQ ID NO: 16 or SEQ ID NO: 17, iii) a polypeptide sequence of SEQ ID NO: 14 and SEQ ID NO: 15 and SEQ ID NO: 16 and SEQ ID NO: 17, or iv) a polypeptide sequence of SEQ ID NO: 18 and SEQ ID NO: 19 and SEQ ID NO: 20 and SEQ ID NO: 21.

In one aspect, the invention provides a PD-1-targeted IL-2 variant immunoconjugate in combination with an anti-TYRP1/anti-CD3 bispecific antibody, wherein the PD-1 targeted IL-2 variant immunoconjugate used in the combination therapy is characterized in comprising the polypeptide sequences of SEQ ID NO: 1, SEQ ID NO: 2, and SEQ ID NO: 3, and wherein the anti-TYRP1/anti-CD3 bispecific antibody used in the combination therapy is characterized in comprising a polypeptide sequence of SEQ ID NO: 14 and SEQ ID NO: 15 and SEQ ID NO: 16 and SEQ ID NO: 17.

In one aspect, the PD-1-targeted IL-2 variant immunoconjugate in combination with an anti-TYRP1/anti-CD3 bispecific antibody is administered to a patient, wherein the patient is treated with or was pre-treated with immunotherapy. Said immunotherapy may comprises adoptive cell transfer, administration of monoclonal antibodies, administration of cytokines, administration of a cancer vaccine, T cell engaging therapies, or any combination thereof. The adoptive cell transfer may comprise administering chimeric antigen receptor expressing T-cells (CAR T-cells), T-cell receptor (TCR) modified T-cells, tumor-infiltrating lymphocytes (TIL), chimeric antigen receptor (CAR)-modified natural killer cells, T cell receptor (TCR) transduced cells, or dendritic cells, or any combination thereof.

### BRIEF DESCRIPTION OF THE FIGURES

FIG. 1. Presents the treatment response rate represented as survival graph of muPD1-IL2v, muFAP-IL2v, muPD-1 and muTYRP1-TCB as single agents and in combination. The B16-muFAP-Fluc double transfectant melanoma cell line was injected intravenous in Black 6 albino mice to study survival in a lung metastatic syngeneic model. The amount of antibodies injected per mouse in mg/kg is the following: 1 mg/kg muPD1-IL2v, 10 mg/kg muPD1, 1.5 mg/kg muFAP-IL2v, and 10 mg/kg muTYRP1-TCB. The antibodies were injected i.v. once weekly for 3 weeks. Significantly superior median and overall survival was observed in the combination 1 mg/kg muPD1-IL2v and 10 mg/kg muTYRP1-TCB group compared to all other single agents, combinations and vehicle groups tested.

FIG. 2. Presents the results of an efficacy experiment comparing muPD1-IL2v, muFAP-IL2v, muPD-1 and muTYRP1-TCB as single agents and in combination. The B16-muFAP-Fluc double transfectant melanoma cell line was injected i.v. in Black 6 albino mice to study survival in a lung metastatic syngeneic model by means of bioluminescence. As early as after the first therapy administration at day 24 a reduction in the B16-muFAP-Fluc bioluminescence signal was detected by IVIS® Spectrum in several TYRP1-TCB treated groups. Only the combination of muPD1-IL2v and muTYRP1-TCB showed a complete disappearance of the BLI signal in all mice that lasted the whole duration of the experiment, indicative of a complete response in 6 out of 6 mice for this combination group.

### DETAILED DESCRIPTION OF THE INVENTION

### IL-2 Pathway

The ability of IL-2 to expand and activate lymphocyte and NK cell populations both in vitro and in vivo explains the anti-tumor effects of IL-2. However, as a regulatory mechanism to prevent excessive immune responses and potential autoimmunity, IL-2 leads to activation-induced cell death (AICD) and renders activated T-cells susceptible to Fas-mediated apoptosis.

Moreover, IL-2 is involved in the maintenance and expansion of peripheral CD4⁺ CD25⁺ T_{reg} cells (Fontenot J D, Rasmussen J P, Gavin M A, et al. A function for interleukin 2 in Foxp3 expressing regulatory T cells. Nat Immunol. 2005; 6:1142-1151; D'Cruz L M, Klein L. Development and function of agonist-induced CD25⁺ Foxp3⁺ regulatory T cells in the absence of interleukin 2 signaling. Nat Immunol. 2005; 6:1152 1159; Maloy K J, Powrie F. Fueling regulation: IL-2 keeps CD4⁺ T_{reg} cells fit. Nat Immunol. 2005; 6:1071-1072). These cells suppress effector T-cells from destroying self or target, either through cell-cell contact or through release of immunosuppressive cytokines, such as IL-10 or transforming growth factor (TGF)-β. Depletion of T_{reg} cells was shown to enhance IL-2-induced anti-tumor immunity (Imai H, Saio M, Nonaka K, et al. Depletion of CD4+CD25+ regulatory T cells enhances interleukin-2-induced antitumor immunity in a mouse model of colon adenocarcinoma. Cancer Sci. 2007; 98:416-423).

IL-2 also plays a significant role in memory CD8+ T-cell differentiation during primary and secondary expansion of CD8+ T cells. IL-2 seems to be responsible for optimal expansion and generation of effector functions following primary antigenic challenge. During the contraction phase of an immune response where most antigen-specific CD8+ T cells disappear by apoptosis, IL-2 signals are able to rescue CD8+ T cells from cell death and provide a durable increase in memory CD8+ T-cells. At the memory stage, CD8+ T-cell frequencies can be boosted by administration of exogenous IL-2. Moreover, only CD8+ T cells that have received IL-2 signals during initial priming are able to mediate efficient secondary expansion following renewed antigenic challenge. Thus, IL-2 signals during different phases of an immune response are key in optimizing CD8+ T-cell functions, thereby affecting both primary and secondary responses of these T cells (Adv Exp Med Biol. 2010; 684:28-41. The role of interleukin-2 in memory CD8 cell differentiation. Boyman O1, Cho J H, Sprent J).

Based on its anti-tumor efficacy, high-dose IL-2 (aldesleukin, marketed as Proleukin®) treatment has been approved for use in patients with metastatic renal cell carcinoma (RCC) and malignant melanoma in the US, and for patients with metastatic RCC in the European Union. However, as a consequence of the mode of action of IL-2, the systemic and untargeted application of IL-2 may considerably compromise anti-tumor immunity via induction of T_{reg} cells and AICD. An additional concern of systemic IL-2 treatment is related to severe side-effects upon intravenous administration, which include severe cardiovascular, pulmonary edema, hepatic, gastrointestinal (GI), neurological, and hematological events (Proleukin (aldesleukin) Summary of Product Characteristics [SmPC]: http://www.medicines.org.uk/emc/medicine/19322/SPC/ (accessed May 27, 2013)). Low-dose IL-2 regimens have been tested in patients, although at the expense of suboptimal therapeutic results. Taken together, therapeutic approaches utilizing IL-2 may be useful for cancer therapy if the liabilities associated with its application can be overcome.

Immunoconjugates comprising a PD-1-targeted antigen binding moiety and an IL-2-based effector moiety, for example including a mutant IL-2, are described in e.g. WO 2018/184964.

In particular, mutant IL-2 (e.g., a quadruple mutant known as IL-2 qm) has been designed to overcome the limitations of wildtype IL-2 (e.g., aldesleukin) or first generation IL-2-based immunoconjugates by eliminating the binding to the IL-2Rα subunit (CD25). This mutant IL-2 qm has been coupled to various tumor-targeting antibodies such as a humanized antibody directed against CEA and an antibody directed against FAP, described in WO 2012/146628 and WO 2012/107417. In addition, the Fc region of the antibody has been modified to prevent binding to Fcγ receptors and the C1q complex. The resulting tumor-targeted IL-2 variant immunoconjugate (e.g., CEA-targeted IL-2 variant immunoconjugate and FAP-targeted IL-2 variant immunoconjugate) have been shown in nonclinical in vitro and in vivo experiments to be able to eliminate tumor cells.

Thus the resulting immunoconjugates represent a class of targeted IL-2 variant immunoconjugates that address the liabilities of IL-2 by eliminating the binding to the IL-2Rα subunit (CD25):

### Properties of Wildtype IL-2 and the IL-2 Variant

| | | IL2v with Eliminated CD25 |
|---|---|---|
| | IL-2 | Binding |
| | Activation of IL-2Rβγ | Activation of IL-2Rβγ |
| | heterodimer and IL-2Rαβγ | heterodimer on effector cells |
| | on effector cells | |
| Advantage | | Reduced sensitivity to |
| | | Fasmediated induction of |
| | | apoptosis (also termed AICD) |
| | | No preferential T_{reg} cells |
| | | stimulation |
| | | No binding to CD25 on lung |
| | | endothelium |
| | | Superior pharmacokinetics and |
| | | targeting (lack of CD25 sink) |
| Dis- | Vascular leak (binding to | |
| advantage | CD25 lung endothelium) | |
| | AICD | |
| | Preferential stimulation of | |
| | T_{reg} cells | |

The term “IL-2” or “human IL-2” refers to the human IL-2 protein including wildtype and variants comprising one or more mutations in the amino acid sequence of wildtype IL-2, for example as shown in SEQ ID NO: 3 having a C125A substitution to avoid the formation of disulphide-bridged IL-2 dimers. IL-2 may also be mutated to remove N- and/or O-glycosylation sites.

### PD-1 Pathway

An important negative co-stimulatory signal regulating T cell activation is provided by programmed death-1 receptor (PD-1)(CD279), and its ligand binding partners PD-L1 (B7-H1, CD274) and PD-L2 (B7-DC, CD273). The negative regulatory role of PD-1 was revealed by PD-1 knock outs (Pdcd1−/−), which are prone to autoimmunity. Nishimura et al., Immunity 11: 141-51 (1999); Nishimura et al., Science 291: 319-22 (2001). PD-1 is related to CD28 and CTLA-4, but lacks the membrane proximal cysteine that allows homodimerization. The cytoplasmic domain of PD-1 contains an immunoreceptor tyrosine-based inhibition motif (ITIM, V/IxYxxL/V). PD-1 only binds to PD-L1 and PD-L2. Freeman et al., J. Exp. Med. 192: 1-9 (2000); Dong et al., Nature Med. 5: 1365-1369 (1999); Latchman et al., Nature Immunol. 2: 261-268 (2001); Tseng et al., J. Exp. Med. 193: 839-846 (2001).

PD-1 can be expressed on T cells, B cells, natural killer T cells, activated monocytes and dendritic cells (DCs). PD-1 is expressed by activated, but not by unstimulated human CD4⁺ and CD8⁺ T cells, B cells and myeloid cells. This stands in contrast to the more restricted expression of CD28 and CTLA-4 (Nishimura et al., Int. Immunol. 8: 773-80 (1996); Boettler et al., J. Virol. 80: 3532-40 (2006)). There are at least 4 variants of PD-1 that have been cloned from activated human T cells, including transcripts lacking (i) exon 2, (ii) exon 3, (iii) exons 2 and 3 or (iv) exons 2 through 4 (Nielsen et al., Cell. Immunol. 235: 109-16 (2005)). With the exception of PD-1 Δex3, all variants are expressed at similar levels as full length PD-1 in resting peripheral blood mononuclear cells (PBMCs). Expression of all variants is significantly induced upon activation of human T cells with anti-CD3 and anti-CD28. The PD-1 Δex3 variants lacks a transmembrane domain, and resembles soluble CTLA-4, which plays an important role in autoimmunity (Ueda et al., Nature 423: 506-11 (2003)). This variant is enriched in the synovial fluid and sera of patients with rheumatoid arthritis. Wan et al., J. Immunol. 177: 8844-50 (2006).

The two PD-1 ligands differ in their expression patterns. PD-L1 is constitutively expressed on mouse T and B cells, CDs, macrophages, mesenchymal stem cells and bone marrow-derived mast cells (Yamazaki et al., J. Immunol. 169: 5538-45 (2002)). PD-L1 is expressed on a wide range of non-hematopoietic cells (e.g., cornea, lung, vascular epithelium, liver non-parenchymal cells, mesenchymal stem cells, pancreatic islets, placental synctiotrophoblasts, keratinocytes, etc.) (Keir et al., Annu. Rev. Immunol. 26: 677-704 (2008)), and is upregulated on a number of cell types after activation. Both type I and type II interferons IFN's) upregulate PD-L1 (Eppihimer et al., Microcirculation 9: 133-45 (2002); Schreiner et al., J. Neuroimmunol. 155: 172-82 (2004)). PD-L1 expression in cell lines is decreased when MyD88, TRAF6 and MEK are inhibited (Liu et al., Blood 110: 296-304 (2007)). JAK2 has also been implicated in PD-L1 induction (Lee et al., FEBS Lett. 580: 755-62 (2006); Liu et al., Blood 110: 296-304 (2007)). Loss or inhibition of phosphatase and tensin homolog (PTEN), a cellular phosphatase that modified phosphatidylinositol 3-kinase (PI3K) and Akt signaling, increased post-transcriptional PD-L1 expression in cancers (Parsa et al., Nat. Med. 13: 84-88 (2007)).

PD-L2 expression is more restricted than PD-L1. PD-L2 is inducibly expressed on DCs, macrophages, and bone marrow-derived mast cells. PD-L2 is also expressed on about half to two-thirds of resting peritoneal B1 cells, but not on conventional B2 B cells (Zhong et al., Eur. J. Immunol. 37: 2405-10 (2007)). PD-L2+B1 cells bind phosphatidylcholine and may be important for innate immune responses against bacterial antigens. Induction of PD-L2 by IFN-gamma is partially dependent upon NF-κB (Liang et al., Eur. J. Immunol. 33: 2706-16 (2003)). PD-L2 can also be induced on monocytes and macrophages by GM-CF, IL-4 and IFN-gamma (Yamazaki et al., J. Immunol. 169: 5538-45 (2002); Loke et al., PNAS 100:5336-41 (2003)).

PD-1 signaling typically has a greater effect on cytokine production than on cellular proliferation, with significant effects on IFN-gamma, TNF-alpha and IL-2 production. PD-1 mediated inhibitory signaling also depends on the strength of the TCR signaling, with greater inhibition delivered at low levels of TCR stimulation. This reduction can be overcome by costimulation through CD28 (Freeman et al., J. Exp. Med. 192: 1027-34 (2000)) or the presence of IL-2 (Carter et al., Eur. J. Immunol. 32: 634-43 (2002)).

Evidence is mounting that signaling through PD-L1 and PD-L2 may be bidirectional. That is, in addition to modifying TCR or BCR signaling, signaling may also be delivered back to the cells expressing PD-L1 and PD-L2. While treatment of dendritic cells with a naturally human anti-PD-L2 antibody isolated from a patient with Waldenstrom's macroglobulinemia was not found to upregulate MHC II or B7 costimulatory molecules, such cells did produce greater amount of proinflammatory cytokines, particularly TNF-alpha and IL-6, and stimulated T cell proliferation (Nguyen et al., J. Exp. Med. 196: 1393-98 (2002)). Treatment of mice with this antibody also (1) enhanced resistance to transplanted b16 melanoma and rapidly induced tumor-specific CTL (Radhakrishnan et al., J. Immunol. 170: 1830-38 (2003); Radhakrishnan et al., Cancer Res. 64: 4965-72 (2004); Heckman et al., Eur. J. Immunol. 37: 1827-35 (2007)); (2) blocked development of airway inflammatory disease in a mouse model of allergic asthma (Radhakrishnan et al., J. Immunol. 173: 1360-65 (2004); Radhakrishnan et al., J. Allergy Clin. Immunol. 116: 668-74 (2005)).

Further evidence of reverse signaling into dendritic cells (“DC's”) results from studies of bone marrow derived DC's cultured with soluble PD-1 (PD-1 EC domain fused to Ig constant region—“s-PD-1”) (Kuipers et al., Eur. J. Immunol. 36: 2472-82 (2006)). This sPD-1 inhibited DC activation and increased IL-10 production, in a manner reversible through administration of anti-PD-1.

Additionally, several studies show a receptor for PD-L1 or PD-L2 that is independent of PD-1. B7.1 has already been identified as a binding partner for PD-L1 (Butte et al., Immunity 27: 111-22 (2007)). Chemical crosslinking studies suggest that PD-L1 and B7.1 can interact through their IgV-like domains. B7.1:PD-L1 interactions can induce an inhibitory signal into T cells. Ligation of PD-L1 on CD4⁺ T cells by B7.1 or ligation of B7.1 on CD4⁺ T cells by PD-L1 delivers an inhibitory signal. T cells lacking CD28 and CTLA-4 show decreased proliferation and cytokine production when stimulated by anti-CD3 plus B7.1 coated beads. In T cells lacking all the receptors for B7.1 (i.e., CD28, CTLA-4 and PD-L1), T cell proliferation and cytokine production were no longer inhibited by anti-CD3 plus B7.1 coated beads. This indicates that B7.1 acts specifically through PD-L1 on the T-cell in the absence of CD28 and CTLA-4. Similarly, T cells lacking PD-1 showed decreased proliferation and cytokine production when stimulated in the presence of anti-CD3 plus PD-L1 coated beads, demonstrating the inhibitory effect of PD-L1 ligation on B7.1 on T cells. When T cells lacking all known receptors for PD-L1 (i.e., no PD-1 and B7.1), T cell proliferation was no longer impaired by anti-CD3 plus PD-L1 coated beads. Thus, PD-L1 can exert an inhibitory effect on T cells either through B7.1 or PD-1.

The direct interaction between B7.1 and PD-L1 suggests that the current understanding of costimulation is incomplete, and underscores the significance to the expression of these molecules on T cells. Studies of PD-L1^{-/-} T cells indicate that PD-L1 on T cells can downregulate T cell cytokine production (Latchman et al., Proc. Natl. Acad. Sci. USA 101: 10691-96 (2004)). Because both PD-L1 and B7.1 are expressed on T cells, B cells, DCs and macrophages, there is the potential for directional interactions between B7.1 and PD-L1 on these cells types. Additionally, PD-L1 on non-hematopoietic cells may interact with B7.1 as well as PD-1 on T cells, raising the question of whether PD-L1 is involved in their regulation. One possible explanation for the inhibitory effect of B7.1:PD-L1 interaction is that T cell PD-L1 may trap or segregate away APC B7.1 from interaction with CD28.

As a result, the antagonism of signaling through PD-L1, including blocking PD-L1 from interacting with either PD-1, B7.1 or both, thereby preventing PD-L1 from sending a negative co-stimulatory signal to T-cells and other antigen presenting cells is likely to enhance immunity in response to infection (e.g., acute and chronic) and tumor immunity. In addition, the anti-PD-L1 antibodies of the present invention, may be combined with antagonists of other components of PD-1:PD-L1 signaling, for example, antagonist anti-PD-1 and anti-PD-L2 antibodies.

The ability of IL-2 to expand and activate lymphocytes and natural killer (NK) cells underlies the anti-tumor activity of IL-2. IL-2 mutants designed to eliminate the binding of IL-2 to IL-2a subunit (CD25) overcome the limitations of IL-2 and as part of a tumor-targeted IL-2 variant immunoconjugate, such as a CEA-targeted IL-2 variant immunoconjugate or a FAP-targeted IL-2 variant immunoconjugate, have been shown to be able to eliminate tumor cells.

### Immunoconjugates and Antibodies

The PD-1-targeted IL-2 variant immunoconjugate used in the combination therapy described herein comprises an antibody which binds to PD-1 on PD-1 expressing immune cells, particularly T cells, or in a tumor cell environment, or an antigen binding fragment thereof, and an IL-2 mutant, particularly a mutant of human IL-2, having reduced binding affinity to the α-subunit of the IL-2 receptor (as compared to wild-type IL-2, e.g. human IL-2 shown as SEQ ID NO: 22), such as an IL-2 comprising: i) one, two or three amino acid substitution(s) at one, two or three position(s) selected from the positions corresponding to residues 42, 45 and 72 of human IL-2 shown as SEQ ID NO: 22, for example three substitutions at three positions, for example the specific amino acid substitutions F42A, Y45A and L72G; or ii) the features as set out in i) plus an amino acid substitution at a position corresponding to residue 3 of human IL-2 shown as SEQ ID NO: 22, for example the specific amino acid substitution T3A; or iii) four amino acid substitutions at positions corresponding to residues 3, 42, 45 and 72 of human IL-2 shown as SEQ ID NO: 22, for example the specific amino acid substitutions T3A, F42A, Y45A and L72G.

The PD-1-targeted IL-2 variant immunoconjugate used in the combination therapy described herein may comprise a heavy chain variable domain and a light chain variable domain of an antibody which binds to PD-1 presented on immune cells, particularly T cells, or in a tumor cell environment and an Fc domain consisting of two subunits and comprising a modification promoting heterodimerization of two non-identical polypeptide chains, and an IL-2 mutant, particularly a mutant of human IL-2, having reduced binding affinity to the α-subunit of the IL-2 receptor (as compared to wild-type IL-2, e.g. human IL-2 shown as SEQ ID NO: 22), such as an IL-2 comprising: i) one, two or three amino acid substitution(s) at one, two or three position(s) selected from the positions corresponding to residues 42, 45 and 72 of human IL-2 shown as SEQ ID NO: 22, for example three substitutions at three positions, for example the specific amino acid substitutions F42A, Y45A and L72G; or ii) the features as set out in i) plus an amino acid substitution at a position corresponding to residue 3 of human IL-2 shown as SEQ ID NO: 22, for example the specific amino acid substitution T3A; or iii) four amino acid substitutions at positions corresponding to residues 3, 42, 45 and 72 of human IL-2 shown as SEQ ID NO: 22, for example the specific amino acid substitutions T3A, F42A, Y45A and L72G.

A PD-1-targeted IL-2 variant immunoconjugate used in the combination therapy may comprise a) a heavy chain variable domain VH of SEQ ID NO: 1 and a light chain variable domain VL of SEQ ID NO: 2, and the polypeptide sequence of SEQ ID NO: 3, or b) a polypeptide sequence of SEQ ID NO: 8 or SEQ ID NO: 9 or SEQ ID NO: 10, or c) the polypeptide sequences of SEQ ID NO: 8, and SEQ ID NO: 9 and SEQ ID NO: 10, or d) the polypeptide sequences of SEQ ID NO: 11, and SEQ ID NO: 12 and SEQ ID NO: 13.

In some embodiments, the PD-1-targeted IL-2 variant immunoconjugate used in the combination therapy comprises the polypeptide sequences of SEQ ID NO: 8, SEQ ID NO: 9 and SEQ ID NO: 10.

These PD-1-targeted IL-2 variant immunoconjugate, along with their component parts of antigen binding moieties, Fc domains and effector moieties, are described as examples of the immunoconjugates described in WO 2018/184964. For example, the particular immunocytokines ‘PD-1-targeted IgG-IL-2 qm fusion protein’ based on the anti-CEA antibody CH1A1A 98/99 2F1 and IL-2 quadruple mutant (qm) are described in e.g., Examples 1 and 2 of WO 2018/184964.

Particular PD-1-targeted IL-2 variant immunoconjugate described in WO 2018/184964 are characterized in comprising the following polypeptide sequences as described herein:

| IL-2 mutant | amino acid sequence, SEQ ID NO: |
|---|---|
| IL-2 qm | 3 |

| | amino acid sequence of | amino acid sequence of |
|---|---|---|
| anti-PD1 | the heavy chain variable | the light chain variable |
| antibody | domain VH, SEQ ID NO: | domain VL, SEQ ID NO: |
| | 1 | 2 |

| | | |
|---|---|---|
| PD-1-targeted | amino acid sequence of | amino acid sequence of |
| IL-2 variant | the heavy chain, SEQ ID | the light chain, SEQ ID |
| immunoconjugate | NOs 8, 9 | NO: 10 |

As described in WO 2012/146628, an IL-2 mutant has reduced binding affinity to the α-subunit of the IL-2 receptor. Together with the β- and γ-subunits (also known as CD122 and CD132, respectively), the α-subunit (also known as CD25) forms the heterotrimeric high affinity IL-2 receptor, while the dimeric receptor consisting only of the β- and γ-subunits is termed the intermediate-affinity IL-2 receptor. As described in WO 2012/146628, an IL-2 mutant polypeptide with reduced binding to the α-subunit of the IL-2 receptor has a reduced ability to induce IL-2 signalling in regulatory T cells, induces less activation-induced cell death (AICD) in T cells, and has a reduced toxicity profile in vivo, compared to a wild-type IL-2 polypeptide. The use of such an IL-2 mutant with reduced toxicity is particularly advantageous in PD-1-targeted IL-2 variant immunoconjugates, having a long serum half-life due to the presence of an Fc domain. The IL-2 mutant may comprise at least one amino acid mutation that reduces or abolishes the affinity of the IL-2 mutant to the α-subunit of the IL-2 receptor (CD25) but preserves the affinity of the IL-2 mutant to the intermediate-affinity IL-2 receptor (consisting of the β- and γ-subunits of the IL-2 receptor), compared to wildtype IL-2. The one or more amino acid mutations may be amino acid substitutions. The IL-2 mutant may comprise one, two or three amino acid substitutions at one, two or three position(s) selected from the positions corresponding to residue 42, 45, and 72 of human IL-2 (shown as SEQ ID NO: 22). The IL-2 mutant may comprise three amino acid substitutions at the positions corresponding to residue 42, 45 and 72 of human IL-2. The IL-2 mutant may be a mutant of human IL-2. The IL-2 mutant may be human IL-2 comprising the amino acid substitutions F42A, Y45A and L72G. The IL-2 mutant may additionally comprise an amino acid mutation at a position corresponding to position 3 of human IL-2, which eliminates the O-glycosylation site of IL-2. Particularly, said additional amino acid mutation is an amino acid substitution replacing a threonine residue by an alanine residue. A particular IL-2 mutant useful in the invention comprises four amino acid substitutions at positions corresponding to residues 3, 42, 45 and 72 of human IL-2 (shown as SEQ ID NO: 22). Specific amino acid substitutions are T3A, F42A, Y45A and L72G. As demonstrated in the Examples of WO 2012/146628, said quadruple mutant IL-2 polypeptide (IL-2 qm) exhibits no detectable binding to CD25, reduced ability to induce apoptosis in T cells, reduced ability to induce IL-2 signaling in T_{reg} cells, and a reduced toxicity profile in vivo. However, it retains ability to activate IL-2 signaling in effector cells, to induce proliferation of effector cells, and to generate IFN-γ as a secondary cytokine by NK cells. The IL-2 mutant according to any of the above descriptions may comprise additional mutations that provide further advantages such as increased expression or stability. For example, the cysteine at position 125 may be replaced with a neutral amino acid such as alanine, to avoid the formation of disulfide-bridged IL-2 dimers. Thus, the IL-2 mutant may comprise an additional amino acid mutation at a position corresponding to residue 125 of human IL-2. Said additional amino acid mutation may be the amino acid substitution C125A. The IL-2 mutant may comprise the polypeptide sequence of SEQ ID NO: 3.

In preferred embodiments, PD-1 targeting of the PD-1-targeted IL-2 variant immunoconjugate may be achieved by targeting PD-1, as described in WO 2018/1184964. PD-1-targeting may be achieved with an anti-PD-1 antibody or an antigen binding fragment thereof. An anti-PD-1 antibody may comprise a heavy chain variable region sequence that is at least about 80%, 85%, 90%, 95%, 96%, 97%, 98%, 99% or 100% identical to the sequence of SEQ ID NO: 1 or a variant thereof that retains functionality. An anti-PD-1 antibody may comprise a light chain variable region sequence that is at least about 80%, 85%, 90%, 95%, 96%, 97%, 98%, 99% or 100% identical to the sequence of SEQ ID NO: 2 or a variant thereof that retains functionality. An anti-PD-1 antibody may comprise a heavy chain variable region sequence that is at least about 80%, 85%, 90%, 95%, 96%, 97%, 98%, 99% or 100% identical to the sequence of SEQ ID NO: 1, or a variant thereof that retains functionality, and a light chain variable region sequence that is at least about 80%, 85%, 90%, 95%, 96%, 97%, 98%, 99% or 100% identical to the sequence of SEQ ID NO: 2, or a variant thereof that retains functionality. An anti-PD-1 antibody may comprise the heavy chain variable region sequence of SEQ ID NO: 1 and the light chain variable region sequence of SEQ ID NO: 2.

The PD-1-targeted IL-2 variant immunoconjugate may comprise a polypeptide sequence selected from the group consisting of SEQ ID NO: 8, SEQ ID NO: 9 and SEQ ID NO: 10, or a variant thereof that retains functionality. The PD-1-targeted IL-2 variant immunoconjugate may comprise a polypeptide sequence wherein a Fab heavy chain specific for PD-1 shares a carboxy-terminal peptide bond with an Fc domain subunit comprising a hole modification. The PD-1-targeted IL-2 variant immunoconjugate may comprise the polypeptide sequence of SEQ ID NO: 8 or SEQ ID NO: 9, or a variant thereof that retains functionality. The PD-1-targeted IL-2 variant immunoconjugate may comprise a Fab light chain specific for PD-1. The PD-1-targeted IL-2 variant immunoconjugate may comprise the polypeptide sequence of SEQ ID NO: 10, or a variant thereof that retains functionality. The polypeptides may be covalently linked, e.g., by a disulfide bond. The Fc domain polypeptide chains may comprise the amino acid substitutions L234A, L235A, and P329G (which may be referred to as LALA P329G).

As described in WO 2018/184964, the PD-1-targeted IL-2 variant immunoconjugate may be a PD-1-targeted IgG-IL-2 qm fusion protein having the sequences shown as SEQ ID NOs: 8, 9 and 10 (as described in e.g. Example 1 of WO 2018/184964). The PD-1-targeted IL-2 variant immunoconjugate having the sequences shown as SEQ ID NOs: 8, 9 and 10 is referred to herein as “PD1-IL2v”. The PD-1-targeted IL-2 variant immunoconjugate having the sequences shown as SEQ ID NOs: 11, 12 and 13 is referred to herein as “muPD1-IL2v”, which is a murine surrogate. The PD-1-targeted IL-2 variant immunoconjugate used in the combination therapy described herein may comprise an antibody which binds to an antigen presented on immune cells, particularly T cells, or in a tumor cell environment, and an IL-2 mutant having reduced binding affinity to the subunit of the IL-2 receptor. The PD-1-targeted IL-2 variant immunoconjugate may essentially consist of an antibody which binds to PD-1 presented on immune cells, particularly T cells, or in a tumor cell environment, and an IL-2 mutant having reduced binding affinity to the subunit of the IL-2 receptor. The antibody may be an IgG antibody, particularly an IgG1 antibody. The PD-1-targeted IL-2 variant immunoconjugate may comprise a single IL-2 mutant having reduced binding affinity to the subunit of the IL-2 receptor (i.e. not more than one IL-2 mutant moiety is present). Anti-TYRP1/anti-CD3 bispecific antibodies are described in WO 2020/127619 A1.

The anti-TYRP1/anti-CD3 bispecific antibody used in the combination therapy described herein comprises a first antigen binding moiety capable of binding TYRP1 and a second antigen binding moiety capable of binding CD3. The anti-TYRP1/anti-CD3 bispecific antibody used in the combination therapy described herein may comprise a first antigen binding moiety comprising a heavy chain variable region sequence that is at least about 80%, 85%, 90%, 95%, 96%, 97%, 98%, 99% or 100% identical to the sequence of SEQ ID NO: 4 or a variant thereof that retains functionality and a light chain variable region sequence that is at least about 80%, 85%, 90%, 95%, 96%, 97%, 98%, 99% or 100% identical to the sequence of SEQ ID NO: 5 or a variant thereof that retains functionality, and a second antigen binding moiety comprising a heavy chain variable region sequence that is at least about 80%, 85%, 90%, 95%, 96%, 97%, 98%, 99% or 100% identical to the sequence of SEQ ID NO: 6 or a variant thereof that retains functionality and a light chain variable region sequence that is at least about 80%, 85%, 90%, 95%, 96%, 97%, 98%, 99% or 100% identical to the sequence of SEQ ID NO: 7 or a variant thereof that retains functionality. The anti-TYRP1/anti-CD3 bispecific antibody used in the combination therapy described herein may comprise a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7.

The anti-TYRP1/anti-CD3 bispecific antibody used in the combination therapy described herein may comprise a third antigen binding moiety which is identical to the first antigen binding moiety. In one embodiment, the first antigen binding moiety and the third antigen moiety which bind to TYRP1 are conventional Fab molecules. In such embodiments, the second antigen binding moiety that binds to CD3 is a crossover Fab molecule, i.e. a Fab molecule wherein the variable domains VH and VL of the Fab heavy and light chains are exchanged/replaced by each other.

The anti-TYRP1/anti-CD3 bispecific antibody used in the combination therapy may comprise amino acid substitutions in Fab molecules comprised therein which are particularly efficient in reducing mispairing of light chains with non-matching heavy chains (Bence-Jones-type side products), which can occur in the production of Fab-based multispecific antibodies with a VH/VL exchange in one (or more, in case of molecules comprising more than two antigen-binding Fab molecules) of their binding arms (see also PCT publication no. WO 2015/150447, particularly the examples therein, incorporated herein by reference in its entirety). The ratio of a desired (multispecific) antibody compared to undesired side products, in particular Bence Jones-type side products occurring in multispecific antibodies with a VH/VL domain exchange in one of their binding arms, can be improved by the introduction of charged amino acids with opposite charges at specific amino acid positions in the CH1 and CL domains (sometimes referred to herein as “charge modifications”).

Accordingly, anti-TYRP1/anti-CD3 bispecific antibodies used in the combination therapy wherein the first and the second (and, where present, third) antigen binding moieties of the (multispecific) antibody are Fab molecules, and in one of the antigen binding moieties (particularly the second antigen binding moiety) the variable domains VL and VH of the Fab light chain and the Fab heavy chain are replaced by each other, in the constant domain CL of the first (and, where present, third) antigen binding moiety the amino acid at position 124 and the amino acid at position 213 may be substituted by a positively charged amino acid (numbering according to Kabat), and wherein in the constant domain CH1 of the first (and, where present, third) antigen binding moiety the amino acid at position 147 and the amino acid at position 213 may be substituted by a negatively charged amino acid (numbering according to Kabat EU index). The constant domains CL and CH1 of the antigen binding moiety having the VH/VL exchange are not replaced by each other (i.e. remain unexchanged). The amino acid at position 124 and the amino acid at position 213 of the constant domain CL may be substituted independently by lysine (K), arginine (R) or histidine (H) (numbering according to Kabat), the amino acid at position 147 and the amino acid at position 213 of the constant domain CH1 may be substituted independently by glutamic acid (E), or aspartic acid (D) (numbering according to Kabat EU index). The anti-TYRP1/anti-CD3 bispecific antibody used in the combination therapy may in the constant domain CL of the first (and, where present, third) antigen binding moiety at the amino acid at position 124 be substituted by lysine (K) (numbering according to Kabat) and the amino acid at position 123 be substituted by arginine (R) (numbering according to Kabat), and in the constant domain CH1 of the first (and, where present, third) antigen binding domain the amino acid at position 147 be substituted by glutamic acid (E) (numbering according to Kabat EU index) and the amino acid at position 213 be substituted by glutamic acid (E) (numbering according to Kabat EU index).

The anti-TYRP1/anti-CD3 bispecific antibody used in the combination therapy described herein may have the sequences shown as SEQ ID NOs: 14, 15, 16 and 17 or a variant thereof that retains functionality.

The anti-TYRP1/anti-CD3 bispecific antibody having the sequences shown as SEQ ID NOs: 14, 15, 16 and 17 is referred to herein as “TYRP1 TCB” or “TYRP1-TCB”. The anti-TYRP1/anti-CD3 bispecific antibody having the sequences shown as SEQ ID NOs: 18, 19, 20 and 21 is referred to herein as “muTYRP1 TCB”, which is a murine surrogate.

As described herein, the PD-1-targeted IL-2 variant immunoconjugate used in the combination therapy described herein may comprise a heavy chain variable domain and a light chain variable domain of an antibody which binds to immune cells, particularly T cells, or in a tumor cell environment and an Fc domain consisting of two subunits and comprising a modification promoting heterodimerization of two non-identical polypeptide chains. The PD-1-targeted IL-2 variant immunoconjugate used in the combination therapy described herein may comprise a heavy chain variable domain of an antibody which binds to immune cells, particularly T cells, or in a tumor cell environment and an Fc domain subunit comprising a knob mutation, a heavy chain variable domain of an antibody which binds to immune cells, particularly T cells, or in a tumor cell environment and an Fc domain subunit comprising a hole mutation, and a light chain variable domain of an antibody which binds to immune cells, particularly T cells, or in a tumor cell environment, and an IL-2 mutant having reduced binding affinity to the subunit of the IL-2 receptor. Thus an immunoconjugate may comprise an Fc domain comprising a modification promoting heterodimerization of two non-identical polypeptide chains. A “modification promoting heterodimerization” is a manipulation of the peptide backbone or the post-translational modifications of a polypeptide that reduces or prevents the association of the polypeptide with an identical polypeptide to form a homodimer. A modification promoting heterodimerization as used herein particularly includes separate modifications made to each of two polypeptides desired to form a dimer, wherein the modifications are complementary to each other so as to promote association of the two polypeptides. For example, a modification promoting heterodimerization may alter the structure or charge of one or both of the polypeptides desired to form a dimer so as to make their association sterically or electrostatically favorable, respectively. Heterodimerization occurs between two non-identical polypeptides, such as two subunits of an Fc domain wherein further immunoconjugate components fused to each of the subunits (e.g. antigen binding moiety, effector moiety) are not the same. In the immunoconjugates according to the present invention, the modification promoting heterodimerization is in the Fc domain. In some embodiments the modification promoting heterodimerziation comprises an amino acid mutation, specifically an amino acid substitution. In a particular embodiment, the modification promoting heterodimerization comprises a separate amino acid mutation, specifically an amino acid substitution, in each of the two subunits of the Fc domain. The site of most extensive protein-protein interaction between the two polypeptide chains of a human IgG Fc domain is in the CH3 domain of the Fc domain. Thus, in one embodiment said modification is in the CH3 domain of the Fc domain. In a specific embodiment said modification is a knob-into-hole modification, comprising a knob modification in one of the two subunits of the Fc domain and a hole modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in formation of a disulfide bridge between the two subunits of the Fc region, further stabilizing the dimer (Carter, J Immunol Methods 248, 7-15 (2001)). Numbering of amino acid residues in the Fc region is according to the EU numbering system, also called the EU index, as described in Kabat et al., Sequences of Proteins of Immunological Interest, 5th Ed. Public Health Service, National Institutes of Health, Bethesda, M D, 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

In an alternative embodiment a modification promoting heterodimerization of two non-identical polypeptide chains comprises a modification mediating electrostatic steering effects, e.g. as described in WO 2009/089004. Generally, this method involves replacement of one or more amino acid residues at the interface of the two polypeptide chains by charged amino acid residues so that homodimer formation becomes electrostatically unfavorable but heterodimerization electrostatically favorable.

An IL-2 mutant having reduced binding affinity to the subunit of the IL-2 receptor may be fused to the carboxy-terminal amino acid of the subunit of the Fc domain comprising the knob modification. Without wishing to be bound by theory, fusion of the IL-2 mutant to the knob-containing subunit of the Fc domain will further minimize the generation of homodimeric immunoconjugate comprising two IL-2 mutant polypeptides (steric clash of two knob-containing polypeptides).

The Fc domain of the immunoconjugate and bispecific antibody may be engineered to have altered binding affinity to an Fc receptor, specifically altered binding affinity to an Fcγ receptor, as compared to a non-engineered Fc domain, as described in WO 2012/146628. Binding of the Fc domain to a complement component, specifically to C1q, may be altered, as described in WO 2012/146628. The Fc domain confers to the immunoconjugate and bispecific antibody favorable pharmacokinetic properties, including a long serum half-life which contributes to good accumulation in the target tissue and a favorable tissue-blood distribution ratio. At the same time it may, however, lead to undesirable targeting to cells expressing Fc receptors rather than to the preferred antigen-bearing cells. Moreover, the co-activation of Fc receptor signaling pathways may lead to cytokine release which, in combination with the effector moiety and the long half-life of the immunoconjugate, results in excessive activation of cytokine receptors and severe side effects upon systemic administration. In line with this, conventional IgG-IL-2 immunoconjugates have been described to be associated with infusion reactions (see e.g. King et al., J Clin Oncol 22, 4463-4473 (2004)).

Accordingly, the Fc domain of the immunoconjugate and bispecific antibody may be engineered to have reduced binding affinity to an Fc receptor. In one such embodiment the Fc domain comprises one or more amino acid mutation that reduces the binding affinity of the Fc domain to an Fc receptor. Typically, the same one or more amino acid mutation is present in each of the two subunits of the Fc domain. In one embodiment said amino acid mutation reduces the binding affinity of the Fc domain to the Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to the Fc receptor by at least 10-fold, at least 20-fold, or even at least 50-fold. In one embodiment the immunoconjugate and bispecific antibody comprising an engineered Fc domain exhibit less than 20%, particularly less than 10%, more particularly less than 5% of the binding affinity to an Fc receptor as compared to immunoconjugates and bispecific antibodies comprising a non-engineered Fc domain. In one embodiment the Fc receptor is an activating Fc receptor. In a specific embodiment the Fc receptor is an Fcγ receptor, more specifically an Fcγ RIIIa, Fcγ RI or Fcγ RIIa receptor. Preferably, binding to each of these receptors is reduced. In some embodiments binding affinity to a complement component, specifically binding affinity to C1q, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or the immunoconjugate comprising said Fc domain) exhibits greater than about 70% of the binding affinity of a non-engineered form of the Fc domain (or the immunoconjugate comprising said non-engineered form of the Fc domain) to FcRn. Fc domains, or immunoconjugates and bispecific antibodies of the invention comprising said Fc domains, may exhibit greater than about 80% and even greater than about 90% of such affinity. In one embodiment the amino acid mutation is an amino acid substitution. In one embodiment the Fc domain comprises an amino acid substitution at position P329. In a more specific embodiment the amino acid substitution is P329A or P329G, particularly P329G. In one embodiment the Fc domain comprises a further amino acid substitution at a position selected from S228, E233, L234, L235, N297 and P331. In a more specific embodiment the further amino acid substitution is S228P, E233P, L234A, L235A, L235E, N297A, N297D or P331S. In a particular embodiment the Fc domain comprises amino acid substitutions at positions P329, L234 and L235. In a more particular embodiment the Fc domain comprises the amino acid mutations L234A, L235A and P329G (LALA P329G). This combination of amino acid substitutions almost completely abolishes Fcγ receptor binding of a human IgG Fc domain, as described in WO 2012/130831, incorporated herein by reference in its entirety. WO 2012/130831 also describes methods of preparing such mutant Fc domains and methods for determining its properties such as Fc receptor binding or effector functions. Numbering of amino acid residues in the Fc region is according to the EU numbering system, also called the EU index, as described in Kabat et al., Sequences of Proteins of Immunological Interest, 5th Ed. Public Health Service, National Institutes of Health, Bethesda, M D, 1991.

Mutant Fc domains can be prepared by amino acid deletion, substitution, insertion or modification using genetic or chemical methods well known in the art and as described in WO 2012/146628. Genetic methods may include site-specific mutagenesis of the encoding DNA sequence, PCR, gene synthesis, and the like. The correct nucleotide changes can be verified for example by sequencing.

In one embodiment the Fc domain is engineered to have decreased effector function, compared to a non-engineered Fc domain, as described in WO 2012/146628. The decreased effector function can include, but is not limited to, one or more of the following: decreased complement dependent cytotoxicity (CDC), decreased antibody-dependent cell-mediated cytotoxicity (ADCC), decreased antibody-dependent cellular phagocytosis (ADCP), decreased cytokine secretion, decreased immune complex-mediated antigen uptake by antigen-presenting cells, decreased binding to NK cells, decreased binding to macrophages, decreased binding to monocytes, decreased binding to polymorphonuclear cells, decreased direct signaling inducing apoptosis, decreased crosslinking of target-bound antibodies, decreased dendritic cell maturation, or decreased T cell priming.

IgG₄ antibodies exhibit reduced binding affinity to Fc receptors and reduced effector functions as compared to IgG₁ antibodies. Hence, in some embodiments the Fc domain of the T cell activating bispecific antigen binding molecules of the invention is an IgG₄ Fc domain, particularly a human IgG₄ Fc domain. In one embodiment the IgG₄ Fc domain comprises amino acid substitutions at position S228, specifically the amino acid substitution S228P. To further reduce its binding affinity to an Fc receptor and/or its effector function, in one embodiment the IgG₄ Fc domain comprises an amino acid substitution at position L235, specifically the amino acid substitution L235E. In another embodiment, the IgG₄ Fc domain comprises an amino acid substitution at position P329, specifically the amino acid substitution P329G. In a particular embodiment, the IgG₄ Fc domain comprises amino acid substitutions at positions S228, L235 and P329, specifically amino acid substitutions S228P, L235E and P329G. Such IgG₄ Fc domain mutants and their Fcγ receptor binding properties are described in European patent application no. WO 2012/130831, incorporated herein by reference in its entirety.

In an embodiment of the invention the PD1-targeted IL-2 variant immunoconjugate used in the combination therapy described herein is characterized in comprising a) a heavy chain variable domain VH of SEQ ID NO: 1 and a light chain variable domain VL of SEQ ID NO: 2, and the polypeptide sequence of SEQ ID NO: 3, or b) a polypeptide sequence of SEQ ID NO: 8 or SEQ ID NO: 9 or SEQ ID NO: 10, or c) the polypeptide sequences of SEQ ID NO: 8, and SEQ ID NO: 9 and SEQ ID NO: 10, or d) the polypeptide sequences of SEQ ID NO: 11, and SEQ ID NO: 12 and SEQ ID NO: 13, and the bispecific antibody which binds to human TYRP1 and CD3 used in the combination therapy is characterized in comprising a) a polypeptide sequence of SEQ ID NO: 14 and SEQ ID NO: 15 and SEQ ID NO: 16 and SEQ ID NO: 17, or b) a polypeptide sequence of SEQ ID NO: 18 and SEQ ID NO: 19 and SEQ ID NO: 20 and SEQ ID NO: 21.

### Definitions

Terms are used herein as generally used in the art, unless otherwise defined in the following.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “antigen binding moiety”, “antigen binding domain” or “antigen-binding portion of an antibody” when used herein refers to the part of an antibody that comprises the area which specifically binds to and is complementary to part or all of an antigen. The term thus refers to the amino acid residues of an antibody which are responsible for antigen-binding. An antigen binding domain may be provided by, for example, one or more antibody variable domains (also called antibody variable regions). Particularly, an antigen binding domain comprises an antibody light chain variable region (VL) and an antibody heavy chain variable region (VH). The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, MD (1991) and/or those residues from a “hypervariable loop”. The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “epitope” denotes a protein determinant of an antigen, such as a CEA or human PD-L1, capable of specifically binding to an antibody. Epitopes usually consist of chemically active surface groupings of molecules such as amino acids or sugar side chains and usually epitopes have specific three dimensional structural characteristics, as well as specific charge characteristics. Conformational and nonconformational epitopes are distinguished in that the binding to the former but not the latter is lost in the presence of denaturing solvents.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991. The Fc domain of an antibody is not involved directly in binding of an antibody to an antigen, but exhibit various effector functions. A “Fc domain of an antibody” is a term well known to the skilled artisan and defined on the basis of papain cleavage of antibodies. Depending on the amino acid sequence of the constant region of their heavy chains, antibodies or immunoglobulins are divided in the classes: IgA, IgD, IgE, IgG and IgM, and several of these may be further divided into subclasses (isotypes), e.g. IgG₁, IgG₂, IgG₃, and IgG₄, IgA₁, and IgA₂. According to the heavy chain constant regions the different classes of immunoglobulins are called α, δ, ε, γ, and μ, respectively. The Fc domain of an antibody is directly involved in ADCC (antibody-dependent cell-mediated cytotoxicity) and CDC (complement-dependent cytotoxicity) based on complement activation, C1q binding and Fc receptor binding. Complement activation (CDC) is initiated by binding of complement factor C1q to the Fc domain of most IgG antibody subclasses. While the influence of an antibody on the complement system is dependent on certain conditions, binding to C1q is caused by defined binding sites in the Fc domain. Such binding sites are known in the state of the art and described e.g. by Boackle, R. J., et al., Nature 282 (1979) 742-743; Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virology 75 (2001) 12161-12168; Morgan, A., et al., Immunology 86 (1995) 319-324; EP 0 307 434. Such binding sites are e.g. L234, L235, D270, N297, E318, K320, K322, P331 and P329 (numbering according to EU index of Kabat, E. A., see above). Antibodies of subclass IgG₁, IgG₂ and IgG₃ usually show complement activation and C1q and C3 binding, whereas IgG₄ do not activate the complement system and do not bind C1q and C3.

As used herein, the term “immunoconjugate” refers to a polypeptide molecule that includes at least one IL-2 molecule and at least one antibody. The IL-2 molecule can be joined to the antibody by a variety of interactions and in a variety of configurations as described herein. In particular embodiments, the IL-2 molecule is fused to the antibody via a peptide linker. Particular immunoconjugates according to the invention essentially consist of one IL-2 molecule and an antibody joined by one or more linker sequences.

By “fused” is meant that the components (e.g. an antibody and an IL-2 molecule) are linked by peptide bonds, either directly or via one or more peptide linkers.

As used herein, the terms “first” and “second” with respect to Fc domain subunits etc., are used for convenience of distinguishing when there is more than one of each type of moiety. Use of these terms is not intended to confer a specific order or orientation of the immunoconjugate unless explicitly so stated.

In one embodiment an antibody component of an immunoconjugate or an antibody described herein comprises an Fc domain derived from human origin and preferably all other parts of the human constant regions. As used herein the term “Fc domain derived from human origin” denotes a Fc domain which is either a Fc domain of a human antibody of the subclass IgG₁, IgG₂, IgG₃ or IgG₄, preferably a Fc domain from human IgG₁ subclass, a mutated Fc domain from human IgG₁ subclass (in one embodiment with a mutation on L234A+L235A), a Fc domain from human IgG₄ subclass or a mutated Fc domain from human IgG₄ subclass (in one embodiment with a mutation on S228P). In one embodiment said antibodies have reduced or minimal effector function. In one embodiment the minimal effector function results from an effectorless Fc mutation. In one embodiment the effectorless Fc mutation is L234A/L235A or L234A/L235A/P329G or N297A or D265A/N297A. In one embodiment the effectorless Fc mutation is selected for each of the antibodies independently of each other from the group comprising (consisting of) L234A/L235A, L234A/L235A/P329G, N297A and D265A/N297A (EU numbering).

In one embodiment the antibody components of immunoconjugates or antibodies described herein are of human IgG class (i.e. of IgG₁, IgG₂, IgG₃ or IgG₄ subclass).

In a preferred embodiment the antibody components of immunoconjugates or antibodies described herein are of human IgG₁ subclass or of human IgG4 subclass. In one embodiment the antibody components of immunoconjugates or antibodies described herein are of human IgG₁ subclass. In one embodiment the antibody components of immunoconjugates or antibodies described herein are of human IgG₄ subclass.

In one embodiment an antibody component of an immunoconjugate or an antibody described herein is characterized in that the constant chains are of human origin. Such constant chains are well known in the state of the art and e.g. described by Kabat, E. A., (see e.g. Johnson, G. and Wu, T. T., Nucleic Acids Res. 28 (2000) 214-218).

The terms “nucleic acid” or “nucleic acid molecule”, as used herein, are intended to include DNA molecules and RNA molecules. A nucleic acid molecule may be single-stranded or double-stranded, but preferably is double-stranded DNA.

The term “amino acid” as used within this application denotes the group of naturally occurring carboxy alpha-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program. By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

A method of producing an immunoconjugate or bispecific antibody described herein is provided, wherein the method comprises culturing a host cell comprising a polynucleotide encoding the immunoconjugate or bispecific antibody, as provided herein, under conditions suitable for expression of the immunoconjugate or bispecific antibody, and recovering the immunoconjugate or bispecific antibody from the host cell (or host cell culture medium).

The components of the immunoconjugate or bispecific antibody are genetically fused to each other. Immunoconjugate or bispecific antibody can be designed such that its components are fused directly to each other or indirectly through a linker sequence. The composition and length of the linker may be determined in accordance with methods well known in the art and may be tested for efficacy. Additional sequences may also be included to incorporate a cleavage site to separate the individual components of the fusion if desired, for example an endopeptidase recognition sequence.

The immunoconjugate and bispecific antibody comprises at least an antibody variable region capable of binding an antigenic determinant. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty). Antigen binding moieties and methods for producing the same are also described in detail in PCT publication WO 2011/020783, the entire content of which is incorporated herein by reference.

Any animal species of antibody, antibody fragment, antigen binding domain or variable region can be used in the immunoconjugates and bispecific antibody described herein. Non-limiting antibodies, antibody fragments, antigen binding domains or variable regions useful in the present invention can be of murine, primate, or human origin. Where the immunoconjugate is intended for human use, a chimeric form of antibody may be used wherein the constant regions of the antibody are from a human. A humanized or fully human form of the antibody can also be prepared in accordance with methods well known in the art (see e. g. U.S. Pat. No. 5,565,332). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N J, 2001); and McCafferty et al., Nature 348, 552-554; Clackson et al., Nature 352, 624-628 (1991)). Phage typically display antibody fragments, either as single-chain Fv (scFv) fragments or as Fab fragments. A detailed description of the preparation of antigen binding moieties for immunoconjugates by phage display can be found in the Examples appended to PCT publication WO 2011/020783.

In certain embodiments, antibodies are engineered to have enhanced binding affinity according to, for example, the methods disclosed in PCT publication WO 2011/020783 (see Examples relating to affinity maturation) or U.S. Pat. Appl. Publ. No. 2004/0132066, the entire contents of which are hereby incorporated by reference. The ability of the immunoconjugate and bispecific antibody to bind to a specific antigenic determinant can be measured either through an enzyme-linked immunosorbent assay (ELISA) or other techniques familiar to one of skill in the art, e.g. surface plasmon resonance technique (analyzed on a BIACORE T100 system) (Liljeblad, et al., Glyco J 17, 323-329 (2000)), and traditional binding assays (Heeley, Endocr Res 28, 217-229 (2002)). Competition assays may be used to identify an antibody, antibody fragment, antigen binding domain or variable domain that competes with a reference antibody for binding to a particular antigen. In certain embodiments, such a competing antibody binds to the same epitope (e.g. a linear or a conformational epitope) that is bound by the reference antibody. Detailed exemplary methods for mapping an epitope to which an antibody binds are provided in Morris (1996) “Epitope Mapping Protocols,” in Methods in Molecular Biology vol. 66 (Humana Press, Totowa, NJ). In an exemplary competition assay, immobilized antigen is incubated in a solution comprising a first labeled antibody that binds to the antigen and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to the antigen. See Harlow and Lane (1988) Antibodies: A Laboratory Manual ch. 14 (Cold Spring Harbor Laboratory, Cold Spring Harbor, NY).

PD-1-targeted IL-2 variant immunoconjugates described herein may be prepared as described in the Examples of WO 2018/184964. Anti-TYRP1/anti-CD3 bispecific antibodies described herein may be prepared as described in the Examples of WO 2020/127619.

Antibodies described herein are preferably produced by recombinant means. Such methods are widely known in the state of the art and comprise protein expression in prokaryotic and eukaryotic cells with subsequent isolation of the antibody polypeptide and usually purification to a pharmaceutically acceptable purity. For the protein expression nucleic acids encoding light and heavy chains or fragments thereof are inserted into expression vectors by standard methods. Expression is performed in appropriate prokaryotic or eukaryotic host cells, such as CHO cells, NS0 cells, SP2/0 cells, HEK293 cells, COS cells, yeast, or E. coli cells, and the antibody is recovered from the cells (from the supernatant or after cells lysis).

Recombinant production of antibodies is well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

The antibodies may be present in whole cells, in a cell lysate, or in a partially purified, or substantially pure form. Purification is performed in order to eliminate other cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987).

Expression in NS0 cells is described by, e.g., Barnes, L. M., et al., Cytotechnology 32 (2000) 109-123; Barnes, L. M., et al., Biotech. Bioeng. 73 (2001) 261-270. Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; Norderhaug, L., et al., J. Immunol. Methods 204 (1997) 77-87. A preferred transient expression system (HEK 293) is described by Schlaeger, E.-J. and Christensen, K., in Cytotechnology 30 (1999) 71-83, and by Schlaeger, E.-J., in J. Immunol. Methods 194 (1996) 191-199.

The heavy and light chain variable domains according to the invention are combined with sequences of promoter, translation initiation, constant region, 3′ untranslated region, polyadenylation, and transcription termination to form expression vector constructs. The heavy and light chain expression constructs can be combined into a single vector, co-transfected, serially transfected, or separately transfected into host cells which are then fused to form a single host cell expressing both chains.

The control sequences that are suitable for prokaryotes, for example, include a promoter, optionally an operator sequence, and a ribosome binding site. Eukaryotic cells are known to utilize promoters, enhancers and polyadenylation signals.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

The monoclonal antibodies are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies are readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant monoclonal antibodies in the host cells.

As used herein, the expressions “cell”, “cell line”, and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included.

### Therapeutic Methods and Compositions

The invention comprises a method for the treatment of a patient in need of therapy, characterized by administering to the patient a therapeutically effective amount of the combination therapy of a PD-1-targeted IL-2 variant immunoconjugate with an anti-TYRP1/anti-CD3 bispecific antibody.

The invention comprises the use of a PD-1-targeted IL-2 variant immunoconjugate with an anti-TYRP1/anti-CD3 bispecific antibody according to the invention for the described combination therapy.

One preferred embodiment of the invention is the combination therapy of a PD-1-targeted IL-2 variant immunoconjugate with an anti-TYRP1/anti-CD3 bispecific antibody of the present invention for use in the treatment of cancer or tumor. Thus one embodiment of the invention is a PD-1-targeted IL-2 variant immunoconjugate described herein for use in the treatment of cancer or tumor in combination with an anti-TYRP1/anti-CD3 antibody as described herein. Another embodiment of the invention is an anti-TYRP1/anti-CD3 antibody described herein for use in the treatment of cancer of tumor in combination with a PD-1-targeted IL-2 variant immunoconjugate as described herein.

The cancer or tumor may present an antigen in a tumor cell environment, e.g. on PD-1+ T cells. PD-1 as the target of the combination therapy may be presented in the tumor cell environment, e.g. in PD-1+ T cells. The treatment may be of a solid tumor. The treatment may be of a carcinoma. The cancer may be selected from the group consisting of colorectal cancer, head and neck cancer, non-small cell lung cancer, breast cancer, pancreatic cancer, liver cancer and gastric cancer. The cancer may be selected from the group consisting of lung cancer, colon cancer, gastric cancer, breast cancer, head and neck cancer, skin cancer, liver cancer, kidney cancer, prostate cancer, pancreatic cancer, brain cancer and cancer of the skeletal muscle.

The term “cancer” as used herein may be, for example, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, lymphocytic leukemia, including refractory versions of any of the above cancers, or a combination of one or more of the above cancers. In one preferred embodiment such cancer is a breast cancer, colorectal cancer, melanoma, head and neck cancer, lung cancer or prostate cancer. In one preferred embodiment such cancer is a breast cancer, ovarian cancer, cervical cancer, lung cancer or prostate cancer. In another preferred embodiment such cancer is breast cancer, lung cancer, colon cancer, ovarian cancer, melanoma cancer, bladder cancer, renal cancer, kidney cancer, liver cancer, head and neck cancer, colorectal cancer, pancreatic cancer, gastric carcinoma cancer, esophageal cancer, mesothelioma, prostate cancer, leukemia, lymphoma, myelomas. In one preferred embodiment such cancer is a TYRP1 expressing cancer.

An embodiment of the invention is a PD-1-targeted IL-2 variant immunoconjugate as described herein in combination with an anti-TYRP1/anti-CD3 bispecific antibody as described herein for use in the treatment of any of the above described cancers or tumors. Another embodiment of the invention is an anti-TYRP1/anti-CD3 bispecific antibody as described herein in combination with a PD-1-targeted IL-2 variant immunoconjugate as described herein for use in the treatment of any of the above described cancers or tumors. The invention comprises the combination therapy with a PD-1-targeted IL-2 variant immunoconjugate as described herein with an anti-TYRP1/anti-CD3 bispecific antibody as described herein for the treatment of cancer. The invention comprises the combination therapy with a PD-1-targeted IL-2 variant immunoconjugate as described herein with an anti-TYRP1/anti-CD3 bispecific antibody as described herein for the prevention or treatment of metastasis.

The invention comprises the combination therapy of a PD-1-targeted IL-2 variant immunoconjugate as described herein with an anti-TYRP1/anti-CD3 bispecific antibody as described herein for use in stimulating an immune response or function, such as T cell activity.

The invention comprises a method for the treatment of cancer in a patient in need thereof, characterized by administering to the patient a PD-1-targeted IL-2 variant immunoconjugate as described herein and an anti-TYRP1/anti-CD3 bispecific antibody as described herein. The invention comprises a method for the prevention or treatment of metastasis in a patient in need thereof, characterized by administering to the patient a PD-1-targeted IL-2 variant immunoconjugate as described herein and an anti-TYRP1/anti-CD3 bispecific antibody being as described herein. The invention comprises a method for stimulating an immune response or function, such as T cell activity, in a patient in need thereof, characterized by administering to the patient a PD-1-targeted IL-2 variant immunoconjugate as described herein and an anti-TYRP1/anti-CD3 bispecific antibody as described herein.

The invention comprises a PD-1-targeted IL-2 variant immunoconjugate as described herein for use in the treatment of cancer in combination with an anti-TYRP1/anti-CD3 bispecific antibody as described herein, or alternatively for the manufacture of a medicament for the treatment of cancer in combination with an anti-TYRP1/anti-CD3 bispecific antibody as described herein.

The invention comprises a PD-1-targeted IL-2 variant immunoconjugate as described herein for use in the prevention or treatment of metastasis in combination with an anti-TYRP1/anti-CD3 bispecific antibody as described herein, or alternatively for the manufacture of a medicament for the prevention or treatment of metastasis in combination with an anti-TYRP1/anti-CD3 bispecific antibody as described herein.

The invention comprises a PD-1-targeted IL-2 variant immunoconjugate as described herein for use in stimulating an immune response or function, such as T cell activity, in combination with an anti-TYRP1/anti-CD3 bispecific antibody as described herein, or alternatively for the manufacture of a medicament for use in stimulating an immune response or function, such as T cell activity, in combination with an anti-TYRP1/anti-CD3 bispecific antibody as described herein.

The invention comprises an anti-TYRP1/anti-CD3 bispecific antibody as described herein for use in the treatment of cancer in combination with a PD-1-targeted IL-2 variant immunoconjugate as described herein, or alternatively for the manufacture of a medicament for the treatment of cancer in combination with a PD-1-targeted IL-2 variant immunoconjugate as described herein.

The invention comprises an anti-TYRP1/anti-CD3 bispecific antibody as described herein for use in the prevention or treatment of metastasis in combination with a PD-1-targeted IL-2 variant immunoconjugate as described herein, or alternatively for the manufacture of a medicament for the prevention or treatment of metastasis in combination with a PD-1-targeted IL-2 variant immunoconjugate as described herein.

The invention comprises an anti-TYRP1/anti-CD3 bispecific antibody as described herein for use in stimulating an immune response or function, such as T cell activity, in combination with a PD-1-targeted IL-2 variant immunoconjugate as described herein, or alternatively for the manufacture of a medicament for use in stimulating an immune response or function, such as T cell activity, in combination with a PD-1-targeted IL-2 variant immunoconjugate as described herein.

In a preferred embodiment of the invention the PD-1-targeted IL-2 variant immunoconjugate used in the above described combination treatments and medical uses of different diseases is a PD-1-targeted IL-2 variant immunoconjugate characterized in comprising the polypeptide sequences of SEQ ID NO: 8, SEQ ID NO: 9 and SEQ ID NO: 10, and the anti-TYRP1/anti-CD3 bispecific antibody used in such combination treatments is characterized in comprising the polypeptide sequences of SEQ ID NO: 14, SEQ ID NO: 15, SEQ ID NO: 16 and SEQ ID NO: 17.

In another aspect, the present invention provides a composition, e.g. a pharmaceutical composition, containing a PD-1-targeted IL-2 variant immunoconjugate as described herein and an antibody which binds to human TYRP1 and CD3, or the antigen-binding portion thereof, as described herein formulated together with a pharmaceutically acceptable carrier.

As used herein, “pharmaceutically acceptable carrier” includes any and all solvents, dispersion media, coatings, antibacterial and antifungal agents, isotonic and absorption/resorption delaying agents, and the like that are physiologically compatible. Preferably, the carrier is suitable for injection or infusion.

A composition of the present invention can be administered by a variety of methods known in the art. As will be appreciated by the skilled artisan, the route and/or mode of administration will vary depending upon the desired results.

Pharmaceutically acceptable carriers include sterile aqueous solutions or dispersions and sterile powders for the preparation of sterile injectable solutions or dispersion. The use of such media and agents for pharmaceutically active substances is known in the art. In addition to water, the carrier can be, for example, an isotonic buffered saline solution.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient (effective amount). The selected dosage level will depend upon a variety of pharmacokinetic factors including the activity of the particular compositions of the present invention employed, or the ester, salt or amide thereof, the route of administration, the time of administration, the rate of excretion of the particular compound being employed, other drugs, compounds and/or materials used in combination with the particular compositions employed, the age, sex, weight, condition, general health and prior medical history of the patient being treated, and like factors well known in the medical arts.

In one aspect the invention provides a kit intended for the treatment of a disease, comprising in the same or in separate containers (a) a PD-1-targeted IL-2 variant immunoconjugate as described herein, and (b) an antibody which binds to human TYRP1 and CD3 as described herein, and optionally further comprising (c) a package insert comprising printed instructions directing the use of the combined treatment as a method for treating the disease. Moreover, the kit may comprise (a) a first container with a composition contained therein, wherein the composition comprises an antibody which binds to human TYRP1 and CD3 as described herein; (b) a second container with a composition contained therein, wherein the composition comprises a PD-1-targeted IL-2 variant immunoconjugate as described herein; and optionally (c) a third container with a composition contained therein, wherein the composition comprises a further cytotoxic or otherwise therapeutic agent. The kit in this embodiment of the invention may further comprise a package insert indicating that the compositions can be used to treat a particular condition. Alternatively, or additionally, the kit may further comprise a third (or fourth) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In one aspect the invention provides a kit intended for the treatment of a disease, comprising (a) a container comprising a PD-1-targeted IL-2 variant immunoconjugate as described herein, and (b) a package insert comprising instructions directing the use of the PD-1-targeted IL-2 variant immunoconjugate in a combination therapy with an anti-TYRP1/anti-CD3 antibody as described herein as a method for treating the disease.

In another aspect the invention provides a kit intended for the treatment of a disease, comprising (a) a container comprising an anti-TYRP1/CD3 antibody as described herein, and (b) a package insert comprising instructions directing the use of the anti-TYRP1/anti-CD3 antibody in a combination therapy with PD-1-targeted IL-2 variant immunoconjugate as described herein as a method for treating the disease.

In a further aspect the invention provides a medicament intended for the treatment of a disease, comprising a PD-1-targeted IL-2 variant immunoconjugate as described herein, wherein said medicament is for use in a combination therapy with an antibody which binds to human TYRP1 and CD3 as described herein and optionally comprises a package insert comprising printed instructions directing the use of the combined treatment as a method for treating the disease.

In still a further aspect the invention provides a medicament intended for the treatment of a disease, comprising an antibody which binds to human TYRP1 and CD3 as described herein, wherein said medicament is for use in a combination therapy with a PD-1-targeted IL-2 variant immunoconjugate as described herein and optionally comprises a package insert comprising printed instructions directing the use of the combined treatment as a method for treating the disease.

The term “a method of treating” or its equivalent, when applied to, for example, cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed to induce an overall beneficial course of action.

The terms “administered in combination with” or “co-administration”, “co-administering”, “combination therapy” or “combination treatment” refer to the administration of the PD-1-targeted IL-2 variant immunoconjugate as described herein and the anti-TYRP1/anti-CD3 bispecific antibody as described herein e.g. as separate formulations/applications (or as one single formulation/application). The co-administration can be simultaneous or sequential in either order, wherein preferably there is a time period while both (or all) active agents simultaneously exert their biological activities. Said active agents are co-administered either simultaneously or sequentially (e.g. intravenous (i.v.)) through a continuous infusion. When both therapeutic agents are co-administered sequentially the dose is administered either on the same day in two separate administrations, or one of the agents is administered on day 1 and the second is co-administered on day 2 to day 7, preferably on day 2 to 4. Thus in one embodiment the term “sequentially” means within 7 days after the dose of the first component, preferably within 4 days after the dose of the first component; and the term “simultaneously” means at the same time. The term “co-administration” with respect to the maintenance doses of PD-1-targeted IL-2 variant immunoconjugate and/or anti-TYRP1/anti-CD3 antibody means that the maintenance doses can be either co-administered simultaneously, if the treatment cycle is appropriate for both drugs, e.g. every week. Or the maintenance doses are co-administered sequentially, for example, doses of PD-1-targeted IL-2 variant immunoconjugate and anti-TYRP1/anti-CD3 antibody are given on alternate weeks.

It is self-evident that the antibodies are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

The amount of co-administration and the timing of co-administration will depend on the type (species, gender, age, weight, etc.) and condition of the patient being treated and the severity of the disease or condition being treated. Said PD-1-targeted IL-2 variant immunoconjugate and/or anti-TYRP1/anti-CD3 antibody are suitably co-administered to the patient at one time or over a series of treatments e.g. on the same day or on the day after or at weekly intervals.

In addition to the PD-1-targeted IL-2 variant immunoconjugate in combination with the anti-TYRP1/anti-CD3 antibody also a chemotherapeutic agent can be administered.

In one embodiment such additional chemotherapeutic agents, which may be administered with PD-1-targeted IL-2 variant immunocytok immunoconjugate ine as described herein and the anti-TYRP1/anti-CD3 antibody as described herein, include, but are not limited to, anti-neoplastic agents including alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); Temodal™ (temozolamide), ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguamne, azathioprine, T-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Therapies targeting epigenetic mechanism including, but not limited to, histone deacetylase inhibitors, demethylating agents (e.g., Vidaza) and release of transcriptional repression (ATRA) therapies can also be combined with the antigen binding proteins. In one embodiment the chemotherapeutic agent is selected from the group consisting of taxanes (like e.g. paclitaxel (Taxol), docetaxel (Taxotere), modified paclitaxel (e.g., Abraxane and Opaxio), doxorubicin, sunitinib (Sutent), sorafenib (Nexavar), and other multikinase inhibitors, oxaliplatin, cisplatin and carboplatin, etoposide, gemcitabine, and vinblastine. In one embodiment the chemotherapeutic agent is selected from the group consisting of taxanes (like e.g. taxol (paclitaxel), docetaxel (Taxotere), modified paclitaxel (e.g. Abraxane and Opaxio). In one embodiment, the additional chemotherapeutic agent is selected from 5-fluorouracil (5-FU), leucovorin, irinotecan, or oxaliplatin. In one embodiment the chemotherapeutic agent is 5-fluorouracil, leucovorin and irinotecan (FOLFIRI). In one embodiment the chemotherapeutic agent is 5-fluorouracil, and oxaliplatin (FOLFOX).

Specific examples of combination therapies with additional chemotherapeutic agents include, for instance, therapies taxanes (e.g., docetaxel or paclitaxel) or a modified paclitaxel (e.g., Abraxane or Opaxio), doxorubicin), capecitabine and/or bevacizumab (Avastin) for the treatment of breast cancer; therapies with carboplatin, oxaliplatin, cisplatin, paclitaxel, doxorubicin (or modified doxorubicin (Caelyx or Doxil)), or topotecan (Hycamtin) for ovarian cancer, the therapies with a multi-kinase inhibitor, MKI, (Sutent, Nexavar, or 706) and/or doxorubicin for treatment of kidney cancer; therapies with oxaliplatin, cisplatin and/or radiation for the treatment of squamous cell carcinoma; therapies with taxol and/or carboplatin for the treatment of lung cancer.

Therefore, in one embodiment the additional chemotherapeutic agent is selected from the group of taxanes (docetaxel or paclitaxel or a modified paclitaxel (Abraxane or Opaxio), doxorubicin, capecitabine and/or bevacizumab for the treatment of breast cancer.

In one embodiment the PD-1-targeted IL-2 variant immunoconjugate and anti-TYRP1/anti-CD3 antibody combination therapy is one in which no chemotherapeutic agents are administered.

The invention comprises also a method for the treatment of a patient suffering from such disease as described herein.

The invention further provides a method for the manufacture of a pharmaceutical composition comprising an effective amount of a PD-1-targeted IL-2 variant immunoconjugate according to the invention as described herein and an anti-TYRP1/anti-CD3 antibody according to the invention as described herein together with a pharmaceutically acceptable carrier and the use of the PD-1-targeted IL-2 variant immunoconjugate and anti-TYRP1/anti-CD3 antibody according to the invention as described herein for such a method.

The invention further provides the use of a PD-1-targeted IL-2 variant immunoconjugate according to the invention as described herein and an anti-TYRP1/anti-CD3 antibody according to the invention as described herein in an effective amount for the manufacture of a pharmaceutical agent, preferably together with a pharmaceutically acceptable carrier, for the treatment of a patient suffering from cancer.

### Cell Therapy

In some embodiments, the immunotherapy is an activation immunotherapy. In some embodiments, immunotherapy is provided as a cancer treatment. In some embodiments, immunotherapy comprises adoptive cell transfer.

In some embodiments, adoptive cell transfer comprises administration of a chimeric antigen receptor-expressing T-cell (CAR T-cell). A skilled artisan would appreciate that CARs are a type of antigen-targeted receptor composed of intracellular T-cell signaling domains fused to extracellular tumor-binding moieties, most commonly single-chain variable fragments (scFvs) from monoclonal antibodies.

CARs directly recognize cell surface antigens, independent of MHC-mediated presentation, permitting the use of a single receptor construct specific for any given antigen in all patients. Initial CARs fused antigen-recognition domains to the CD3 activation chain of the T-cell receptor (TCR) complex. While these first-generation CARs induced T-cell effector function in vitro, they were largely limited by poor antitumor efficacy in vivo. Subsequent CAR iterations have included secondary costimulatory signals in tandem with CD3, including intracellular domains from CD28 or a variety of TNF receptor family molecules such as 4-1BB (CD137) and OX40 (CD134). Further, third generation receptors include two costimulatory signals in addition to CD3, most commonly from CD28 and 4-1BB. Second and third generation CARs dramatically improve antitumor efficacy, in some cases inducing complete remissions in patients with advanced cancer. In one embodiment, a CAR T-cell is an immunoresponsive cell modified to express CARs, which is activated when CARs bind to its antigen.

In one embodiment, a CAR T-cell is an immunoresponsive cell comprising an antigen receptor, which is activated when its receptor binds to its antigen. In one embodiment, the CAR T-cells used in the compositions and methods as disclosed herein are first generation CAR T-cells. In another embodiment, the CAR T-cells used in the compositions and methods as disclosed herein are second generation CAR T-cells. In another embodiment, the CAR T-cells used in the compositions and methods as disclosed herein are third generation CAR T-cells. In another embodiment, the CAR T-cells used in the compositions and methods as disclosed herein are fourth generation CAR T-cells.

In some embodiments, adoptive cell transfer comprises administering T-cell receptor (TCR) modified T-cells. A skilled artisan would appreciate that TCR modified T-cells are manufactured by isolating T-cells from tumor tissue and isolating their TCRa and TCRβ chains. These TCRa and TCRβ are later cloned and transfected into T cells isolated from peripheral blood, which then express TCRa and TCRβ from T-cells recognizing the tumor.

In some embodiments, adoptive cell transfer comprises administering tumor infiltrating lymphocytes (TIL). In some embodiments, adoptive cell transfer comprises administering chimeric antigen receptor (CAR)-modified NK cells. A skilled artisan would appreciate that CAR-modified NK cells comprise NK cells isolated from the patient or commercially available NK engineered to express a CAR that recognizes a tumor-specific protein.

In some embodiments, adoptive cell transfer comprises administering dendritic cells.

In some embodiments, immunotherapy comprises administering of a cancer vaccine. A skilled artisan would appreciate that a cancer vaccine exposes the immune system to a cancer-specific antigen and an adjuvant. In some embodiments, the cancer vaccine is selected from a group comprising: sipuleucel-T, GVAX, ADXS11-001, ADXS31-001, ADXS31-164, ALVAC-CEA vaccine, AC Vaccine, talimogene laherparepvec, BiovaxID, Prostvac, CDX110, CDX1307, CDX1401, CimaVax-EGF, CV9104, DNDN, NeuVax, Ae-37, GRNVAC, tarmogens, GI-4000, GI-6207, GI-6301, ImPACT Therapy, IMA901, hepcortespenlisimut-L, Stimuvax, DCVax-L, DCVax-Direct, DCVax Prostate, CBLI, Cvac, RGSH4K, SCIB1, NCT01758328, and PVX-410. The following examples, sequence listing and figures are provided to aid the understanding of the present invention, the true scope of which is set forth in the appended claims. It is understood that modifications can be made in the procedures set forth without departing from the spirit of the invention.

In the following statements, embodiments of the invention are described:
1. A PD-1-targeted IL-2 variant immunoconjugate in combination with an anti-TYRP1/anti-CD3 bispecific antibody for use as a combination therapy in the treatment of cancer, for use as a combination therapy in the prevention or treatment of metastasis, for use as a combination therapy in treating or delaying progression of an immune related disease such as tumor immunity, or for use as a combination therapy in stimulating an immune response or function, such as T cell activity, wherein the PD-1-targeted IL-2 variant immunoconjugate comprises a heavy chain variable domain VH of SEQ ID NO: 1 and a light chain variable domain VL of SEQ ID NO: 2, and the polypeptide sequence of SEQ ID NO: 3, and wherein the anti-TYRP1/anti-CD3 bispecific antibody comprises a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7.
2. The PD-1-targeted IL-2 variant immunoconjugate in combination with an anti-TYRP1/anti-CD3 bispecific antibody according to the preceding embodiment, for use in the treatment of breast cancer, lung cancer, colon cancer, ovarian cancer, melanoma cancer, bladder cancer, renal cancer, kidney cancer, liver cancer, head and neck cancer, colorectal cancer, melanoma, pancreatic cancer, gastric carcinoma cancer, esophageal cancer, mesothelioma, prostate cancer, leukemia, lymphomas, myelomas.
3. The PD-1-targeted IL-2 variant immunoconjugate in combination with an anti-TYRP1/anti-CD3 bispecific antibody according to any preceding embodiments, characterized in that the antibody component of the immunoconjugate and the antibody are of human IgG₁ or human IgG₄ subclass.
4. The PD-1-targeted IL-2 variant immunoconjugate in combination with an anti-TYRP1/anti-CD3 bispecific antibody according to any one of the preceding embodiments, characterized in that said antibodies have reduced or minimal effector function.
5. The PD-1-targeted IL-2 variant immunoconjugate in combination with an anti-TYRP1/anti-CD3 bispecific antibody according to any one of the preceding embodiments, wherein the minimal effector function results from an effectorless Fc mutation.
6. The PD-1-targeted IL-2 variant immunoconjugate in combination with an anti-TYRP1/anti-CD3 bispecific antibody according to any one of the preceding embodiments, wherein the effectorless Fc mutation is L234A/L235A or L234A/L235A/P329G or N297A or D265A/N297A.
7. A PD-1-targeted IL-2 variant immunoconjugate in combination with an anti-TYRP1/anti-CD3 bispecific antibody according to any one of the preceding embodiments, wherein the PD-1-targeted IL-2 variant immunoconjugate comprises
   i) a polypeptide sequence of SEQ ID NO: 8 or SEQ ID NO: 9 or SEQ ID NO: 10,
   ii) the polypeptide sequence of SEQ ID NO: 8, and SEQ ID NO: 9 and SEQ ID NO: 10, or
   iii) the polypeptide sequence of SEQ ID NO: 11 and SEQ ID NO: 12 and SEQ ID NO: 13
   and wherein the anti-TYRP1/anti-CD3 bispecific antibody comprises a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7.
8. A PD-1-targeted IL-2 variant immunoconjugate in combination with an anti-TYRP1/anti-CD3 bispecific antibody according to any one of the preceding embodiments, wherein the PD-1-targeted IL-2 variant immunoconjugate comprises
   i) a polypeptide sequence of SEQ ID NO: 8 or SEQ ID NO: 9 or SEQ ID NO: 10,
   ii) the polypeptide sequence of SEQ ID NO: 8, and SEQ ID NO: 9 and SEQ ID NO: 10, or
   iii) the polypeptide sequence of SEQ ID NO: 11 and SEQ ID NO: 12 and SEQ ID NO: 13,
   and wherein the anti-TYRP1/anti-CD3 bispecific antibody comprises
   i) a polypeptide sequence of SEQ ID NO: 14 or SEQ ID NO: 15 or SEQ ID NO: 16 or SEQ ID NO: 17,
   ii) a polypeptide sequence of SEQ ID NO: 14 and SEQ ID NO: 15 and SEQ ID NO: 16 and SEQ ID NO: 17, or
   iii) a polypeptide sequence of SEQ ID NO: 18 and SEQ ID NO: 19 and SEQ ID NO: 20 and SEQ ID NO: 21.
9. A PD-1-targeted IL-2 variant immunoconjugate in combination with an anti-TYRP1/anti-CD3 bispecific antibody for use in
   i) Inhibition of tumor growth in a tumor; and/or
   ii) Enhancing median and/or overall survival of subjects with a tumor;
   wherein PD-1 is presented on immune cells, particularly T cells, or in a tumor cell environment,
   wherein the PD-1 targeted IL-2 variant immunoconjugate used in the combination therapy is characterized in comprising
   i) a heavy chain variable domain VH of SEQ ID NO: 1 and a light chain variable domain VL of SEQ ID NO: 2, and the polypeptide sequence of SEQ ID NO: 3,
   ii) a polypeptide sequence of SEQ ID NO: 8 or SEQ ID NO: 9 or SEQ ID NO: 10,
   iii) the polypeptide sequence of SEQ ID NO: 8, and SEQ ID NO: 9 and SEQ ID NO: 10, or
   iv) the polypeptide sequence of SEQ ID NO: 11 and SEQ ID NO: 12 and SEQ ID NO: 12,
   and the anti-TYRP1/anti-CD3 bispecific antibody used in the combination therapy is characterized in comprising
   i) a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7;
   ii) a polypeptide sequence of SEQ ID NO: 14 or SEQ ID NO: 15 or SEQ ID NO: 16 or SEQ ID NO: 17,
   iii) a polypeptide sequence of SEQ ID NO: 14 and SEQ ID NO: 15 and SEQ ID NO: 16 and SEQ ID NO: 17, or
   iv) a polypeptide sequence of SEQ ID NO: 18 and SEQ ID NO: 19 and SEQ ID NO: 20 and SEQ ID NO: 21.
10. A PD-1-targeted IL-2 variant immunoconjugate in combination with an anti-TYRP1/anti-CD3 bispecific antibody according to any one of the preceding embodiments, wherein the PD-1 targeted IL-2 variant immunoconjugate used in the combination therapy is characterized in comprising the polypeptide sequences of SEQ ID NO: 1, SEQ ID NO: 2, and SEQ ID NO: 3, and wherein the anti-TYRP1/anti-CD3 bispecific antibody used in the combination therapy is characterized in comprising a polypeptide sequence of SEQ ID NO: 14 and SEQ ID NO: 15 and SEQ ID NO: 16 and SEQ ID NO: 17.
11. The PD-1-targeted IL-2 variant immunoconjugate in combination with an anti-TYRP1/anti-CD3 bispecific antibody according to any one of the preceding embodiments, wherein the patient is treated with or was pre-treated with immunotherapy.
12. The PD-1-targeted IL-2 variant immunoconjugate in combination with an anti-TYRP1/anti-CD3 bispecific antibody according to the preceding embodiment, wherein said immunotherapy comprises adoptive cell transfer, administration of monoclonal antibodies, administration of cytokines, administration of a cancer vaccine, T cell engaging therapies, or any combination thereof.
13. The PD-1-targeted IL-2 variant immunoconjugate in combination with an anti-TYRP1/anti-CD3 bispecific antibody according to the preceding embodiment, wherein the adoptive cell transfer comprises administering chimeric antigen receptor expressing T-cells (CAR T-cells), T-cell receptor (TCR) modified T-cells, tumor-infiltrating lymphocytes (TIL), chimeric antigen receptor (CAR)-modified natural killer cells, T cell receptor (TCR) transduced cells, or dendritic cells, or any combination thereof.
14. A therapeutic method comprising administering to a subject a combination therapy comprising (a) a PD-1-targeted IL-2 variant immunoconjugate in combination with (b) an anti-TYRP1/anti-CD3 bispecific antibody, wherein the combination therapy is for treating a cancer, for prevention or treatment of metastasis, for treating or delaying progression of an immune related disease such as tumor immunity, or for stimulating an immune response or function, such as T cell activity, wherein the PD-1-targeted IL-2 variant immunoconjugate comprises a heavy chain variable domain VH of SEQ ID NO: 1 and a light chain variable domain VL of SEQ ID NO: 2, and the polypeptide sequence of SEQ ID NO: 3, and wherein the anti-TYRP1/anti-CD3 bispecific antibody comprises a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7.
15. The method of the preceding embodiment, wherein the cancer is selected from the group consisting of breast cancer, lung cancer, colon cancer, ovarian cancer, melanoma cancer, bladder cancer, renal cancer, kidney cancer, liver cancer, head and neck cancer, colorectal cancer, melanoma, pancreatic cancer, gastric carcinoma cancer, esophageal cancer, mesothelioma, prostate cancer, leukemia, lymphomas, and myelomas.
16. The method of the preceding embodiments, wherein the antibody component of the immunoconjugate and the antibody are of human IgG₁ or human IgG₄ subclass.
17. The method of the preceding embodiments, wherein the antibodies have reduced or minimal effector function.
18. The method of the preceding embodiments, wherein the minimal effector function results from an effectorless Fc mutation.
19. The method of the preceding embodiments, wherein the effectorless Fc mutation is L234A/L235A or L234A/L235A/P329G or N297A or D265A/N297A.
20. The method according to any one of the preceding embodiments, wherein the PD-1-targeted IL-2 variant immunoconjugate comprises
   i) a polypeptide sequence of SEQ ID NO: 8 or SEQ ID NO: 9 or SEQ ID NO: 10,
   ii) the polypeptide sequence of SEQ ID NO: 8, and SEQ ID NO: 9 and SEQ ID NO: 10, or
   iii) the polypeptide sequence of SEQ ID NO: 11 and SEQ ID NO: 12 and SEQ ID NO: 13
   and wherein the anti-TYRP1/anti-CD3 bispecific antibody comprises a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7.
21. The method according to any one of the preceding embodiments, wherein the PD-1-targeted IL-2 variant immunoconjugate comprises
   iv) a polypeptide sequence of SEQ ID NO: 8 or SEQ ID NO: 8 or SEQ ID NO: 10,
   v) the polypeptide sequence of SEQ ID NO: 8, and SEQ ID NO: 9 and SEQ ID NO: 10, or
   vi) the polypeptide sequence of SEQ ID NO: 11 and SEQ ID NO: 12 and SEQ ID NO: 13, and wherein the anti-TYRP1/anti-CD3 bispecific antibody comprises
   iv) a polypeptide sequence of SEQ ID NO: 14 or SEQ ID NO: 15 or SEQ ID NO: 16 or SEQ ID NO: 17,
   v) a polypeptide sequence of SEQ ID NO: 14 and SEQ ID NO: 15 and SEQ ID NO: 16 and SEQ ID NO: 17, or
   vi) a polypeptide sequence of SEQ ID NO: 18 and SEQ ID NO: 18 and SEQ ID NO: 20 and SEQ ID NO: 21.
22. A therapeutic method for (i) inhibiting tumor growth in a tumor; and/or (ii) enhancing median and/or overall survival of subjects with a tumor, wherein PD-1 is presented on immune cells, particularly T cells, or in a tumor cell environment, the method comprising administering to a subject a combination therapy comprising (a) PD-1-targeted IL-2 variant immunoconjugate in combination with (b) an anti-TYRP1/anti-CD3 bispecific antibody,
   wherein the PD-1 targeted IL-2 variant immunoconjugate comprises:
      v) a heavy chain variable domain VH of SEQ ID NO: 1 and a light chain variable domain VL of SEQ ID NO: 2, and the polypeptide sequence of SEQ ID NO: 3,
      vi) a polypeptide sequence of SEQ ID NO: 8 or SEQ ID NO: 9 or SEQ ID NO: 10,
      vii) the polypeptide sequence of SEQ ID NO: 8, and SEQ ID NO: 9 and SEQ ID NO: 10, or
      viii) the polypeptide sequence of SEQ ID NO: 11 and SEQ ID NO: 12 and SEQ ID NO: 13,
   and wherein the anti-TYRP1/anti-CD3 bispecific antibody comprises:
      v) a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7;
      vi) a polypeptide sequence of SEQ ID NO: 14 or SEQ ID NO: 15 or SEQ ID NO: 16 or SEQ ID NO: 17,
      vii) a polypeptide sequence of SEQ ID NO: 14 and SEQ ID NO: 15 and SEQ ID NO: 16 and SEQ ID NO: 17, or
      viii) a polypeptide sequence of SEQ ID NO: 18 and SEQ ID NO: 19 and SEQ ID NO: 20 and SEQ ID NO: 21.
23. The method of embodiment 22, wherein the PD-1 targeted IL-2 variant immunoconjugate comprises the polypeptide sequences of SEQ ID NO: 1, SEQ ID NO: 2, and SEQ ID NO: 3, and wherein the anti-TYRP1/anti-CD3 bispecific antibody comprises a polypeptide sequence of SEQ ID NO: 14 and SEQ ID NO: 15 and SEQ ID NO: 16 and SEQ ID NO: 17.
24. The method of embodiment 23, wherein the patient is treated with or was pre-treated with immunotherapy.
25. The method of embodiment 24, wherein said immunotherapy comprises adoptive cell transfer, administration of monoclonal antibodies, administration of cytokines, administration of a cancer vaccine, T cell engaging therapies, or any combination thereof.
26. The method of embodiment 25, wherein the adoptive cell transfer comprises administering chimeric antigen receptor expressing T-cells (CAR T-cells), T-cell receptor (TCR) modified T-cells, tumor-infiltrating lymphocytes (TIL), chimeric antigen receptor (CAR)-modified natural killer cells, T cell receptor (TCR) transduced cells, or dendritic cells, or any combination thereof.

### Example

### B16-muFAP-Fluc Metastatic Melanoma Syngeneic Model

In vivo Efficacy of a PD1-IL2v immunoconjugate alone and in combination with an anti-TYRP1/anti-CD3 bispecific antibody (TYRP1-TCB) were tested for anti-tumoral efficacy in a syngeneic model of Mouse Tumor Cell Line. The murine surrogate PD1-IL2v immunoconjugate (SEQ ID NOs 11, 12 and 13), murine surrogate TYRP1-TCB (SEQ ID NO: 18, 19, 20 and 21), the murine surrogate FAP-IL2v (SEQ ID NOs 24, 25 and 26) and murine surrogate anti-PD1 (SEQ ID NOs 27 and 28) were tested in the mouse melanoma B16-muFAP-Fluc double transfectant cell line intravenously injected into Black 6 albino.

B16 cells (mouse melanoma) were originally obtained from ATCC (Manassas, VA, USA) and after expansion deposited in the Roche-Glycart internal cell bank. B16-muFAP-Fluc cell line was produced in house by calcium transfection and sub-cloning techniques. B16-muFAP-Fluc was cultured in RPMI medium containing 10% FCS (Sigma), 200 μg/ml Zeocin, 0.75 μg/ml Puromycin and 1% of Glutamax. The cells were cultured at 37° ° C. in a water-saturated atmosphere at 5% CO₂. Passage 13 was used for transplantation. Cell viability was 96.1%. 2×10⁵ cells per animal were injected intravenously using a 0.3 ml tuberculin syringe (BD Biosciences, Germany). Two hundred microliters (2×10⁵ B16-muFAP-Fluc cells in RPMI medium) cell suspension was injected in the tail vein.

Female Black 6 albino mice aged 10-12 weeks at the start of the experiment (Charles Rivers, Lyon, France) were maintained under specific-pathogen-free conditions with daily cycles of 12 h light/12 h darkness according to committed guidelines (GV-Solas; Felasa; TierschG). The experimental study protocol was reviewed and approved by local government (ZH193/2014). After arrival, animals were maintained for one week to get accustomed to the new environment and for observation. Continuous health monitoring was carried out on a regular basis.

Mice were injected intravenously on study day 0 with 2×10⁵ B16-muFAP-Fluc cells, randomized and weighed. Eighteen days after the tumor cell injection mice were injected i.v. once weekly for 3 weeks with muPD1-IL2v, muPD1 or muTYRP1-TCB single agents and compare to the combination of muPD1-IL2v+muTYRP1-TCB, muFAP-IL2v+muPD1, muTYRP1-TCB+muPD1 and muFAP-IL2v+muPD1+muTYRP1-TCB.

All mice were injected i.v. with 200 μl of the appropriate solution. The mice in the vehicle group were injected with Histidine Buffer and the treatment groups with the muPD1-IL2v at 1 mg/kg, muPD1 at 10 mg/kg, muFAP-IL2v at 1.5 mg/kg and muTYRP1-TCB at 10 mg/kg. To obtain the desired amount of immune-conjugates per 200 μl, the stock solutions were diluted with Histidine Buffer when necessary.

TABLE 1

| | | | Concentration |
|---|---|---|---|
| Compound | Dose | Formulation buffer | (mg/mL) |
| muPD1-IL2v | 20 μg | 20 mM Histidine, | 3.63 |
| P1AD4866-002 | | 140 mM NaCl; pH 6.0 | (=stock solution) |
| (ID8251) | | | |
| muTYRP1-TCB | 200 μg | 20 mM Histidine, | 6.46 |
| P1AD8683-006 | | 140 mM NaCl, | (=stock solution) |
| | | 0.01% Tween20; pH 6.0 | |
| muPD1 | 200 μg | 20 mM Histidine, | 5.84 |
| P1AD4100-002 | | 140 mM NaCl, | (=stock solution) |
| (ID7452) | | 0.01% Tween20; pH 6.0 | |
| muFAP-IL2v | 30 μg | 20 mM Histidine, | 1.42 |
| P1AD4096-002 | | 140 mM NaCl, | (=stock solution) |
| (ID7447) | | 0.01% Tween20; pH 6.0 | |

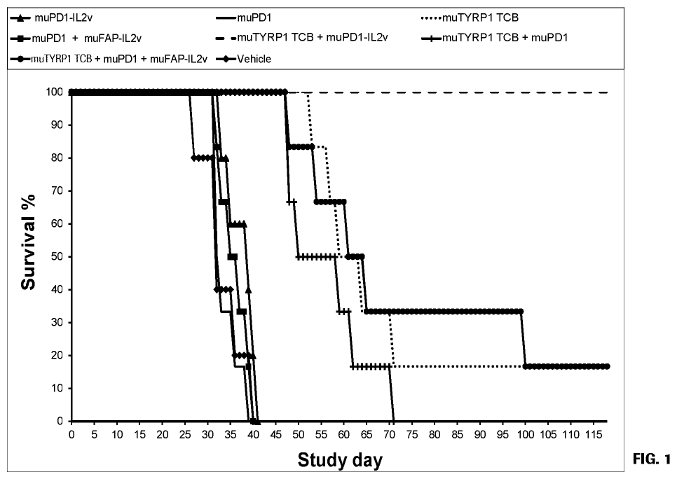

FIG. 1 and Table 2 show that the combination of muPD1-IL2v and muTYRP1-TCB mediates a significantly superior efficacy in terms of enhanced median and overall survival compared to all other treatment and vehicle groups. A Pairwise Log-Rank Test (multiple test level=0.00179) was used for statistical analysis. The Chi-Square value was 66.0720, with a Prob>ChiSq of <0.0001*.

TABLE 2
Median and overall survival of mice of different treatment groups.

| | Median | p-value | |
|---|---|---|---|
| | Survival | vs | Overall |
| Groups | in days | control | survival |
| Vehicle | 32 | 1.0000 | 0/6 |
| 1 mg/kg muPD-1-IL2v | 39 | 0.2064 | 0/6 |
| 10 mg/kg muPD1 + 1.5 mg/kg | 36 | 0.5883 | 0/6 |
| muFAP-IL2v | | | |
| 10 mg/kg muPD1 | 32 | 0.7880 | 0/6 |
| 10 mg/kg muTYRP1-TCB | 61 | 0.0007*** | 1/6 |
| 10 mg/kg muTYRP1-TCB + 10 mg/kg | 54 | 0.0007*** | 0/6 |
| muPD1 | | | |
| 10 mg/kg muTYRP1-TCB + | 63 | 0.0007*** | 1/6 |
| 10 mg/kg muPD1 + 1.5 mg/kg | | | |
| muFAP-IL2v | | | |
| 10 mg/kg muTYRP1-TCB + | Not | <0.0001*** | 6/6 |
| 1 mg/kg muPD-1-IL2v | reached | | |

For Bioluminescence imaging by IVIS® SPECTRUM, the mice are injected subcutaneously with 150 mg/kg of D-Luciferin 10 minutes before bioluminescence imaging acquisition (BLI) and later anesthetized with 4% isoflurane. Subsequently the mice are transferred into an isolation chamber, which is positioned in the IVIS® spectrum. In vivo BLI acquisitions are performed by acquiring the luminescence signal for 10-50 seconds. Data is stored as Radiance (photons)/sec/cm²/sr. In vivo BLI data analysis is performed with the Living Image® 4.4 software and represented by a tumor inhibition curve.

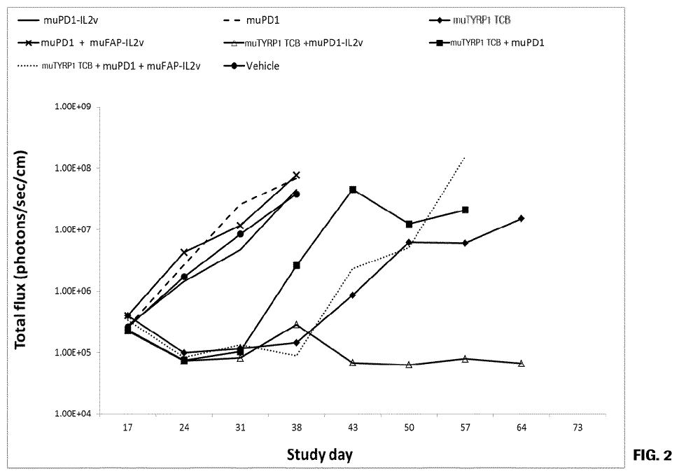

FIG. 2 shows that the combination of muPD1-IL2v+muTYRP1-TCB mediated superior efficacy in terms of decreasing the bioluminescence signal (photons/second) compared to all other single agent, vehicle and combination groups. The decrease in bioluminescence signal is a readout of decrease in tumor burden because the tumor cells are engineered to express luciferase.

Sequences

| | | SEQ ID |
|---|---|---|
| Description | Sequence | NO |
| anti-PD-1 | EVQLLESGGGLVQPGGSLRLSCAASGFSFSSYTMSWVRQAPG | 1 |
| VH | KGLEWVATISGGGRDIYYPDSVKGRFTISRDNSKNTLYLQMN | |
| | SLRAEDTAVYYCVLLTGRVYFALDSWGQGTLVTVSS | |
| anti-PD-1 | DIVMTQSPDSLAVSLGERATINCKASESVDTSDNSFIHWYQQ | 2 |
| VL | KPGQSPKLLIYRSSTLESGVPDRFSGSGSGTDFTLTISSLQA | |
| | EDVAVYYCQQNYDVPWTFGQGTKVEIK | |
| quadruple | APASSSTKKTQLQLEHLLLDLQMILNGINNYKNPKLTRMLTA | 3 |
| mutant | KFAMPKKATELKHLQCLEEELKPLEEVLNGAQSKNFHLRPRD | |
| human IL-2 | LISNINVIVLELKGSETTFMCEYADETATIVEFLNRWITFAQ | |
| (IL-2 qm or | SIISTLT | |
| IL2v) | | |
| anti-TYRP1 | QVQLVQSGAEVKKPGASVKVSCKASGFNIKDYFLHWVRQAPG | 4 |
| VH | QGLEWMGWINPDNGNTVYAQKFQGRVTMTADTSTSTVYMELS | |
| | SLRSEDTAVYYCTRRDYTYEKAALDYWGQGTLVTVSS | |
| anti-TYRP1 | DIQMTQSPSSLSASVGDRVTITCRASGNIYNYLAWYQQKPGK | 5 |
| VL | VPKLLIYDAKTLADGVPSRFSGSGSGTDFTLTISSLQPEDVA | |
| | TYYCQHFWSLPFTFGQGTKLEIK | |
| anti-CD3 VH | EVQLLESGGGLVQPGGSLRLSCAASGFQFSSYAMNWVRQAPG | 6 |
| | KGLEWVSRIRSKYNNYATYYADSVKGRFTISRDDSKNTLYLQ | |
| | MNSLRAEDTAVYYCVRHTTFPSSYVSYYGYWGQGTLVTVSS | |
| anti-CD3 VL | QAVVTQEPSLTVSPGGTVTLTCGSSTGAVTTSNYANWVQEKP | 7 |
| | GQAFRGLIGGTNKRAPGTPARFSGSLLGGKAALTLSGAQPED | |
| | EAEYYCALWYSNLWVFGGGTKLTVL | |
| PD-1 IL2v - | EVQLLESGGGLVQPGGSLRLSCAASGFSFSSYTMSWVRQAPG | 8 |
| HC with | KGLEWVATISGGGRDIYYPDSVKGRFTISRDNSKNTLYLQMN | |
| IL2v (Fc | SLRAEDTAVYYCVLLTGRVYFALDSWGQGTLVTVSSASTKGP | |
| knob, | SVFPLAPSSKSTSGGTAALGCLVKDYFPEPVTVSWNSGALTS | |
| LALAPG) | GVHTFPAVLQSSGLYSLSSVVTVPSSSLGTQTYICNVNHKPS | |
| | NTKVDKKVEPKSCDKTHTCPPCPAPEAAGGPSVFLFPPKPKD | |
| | TLMISRTPEVTCVVVDVSHEDPEVKFNWYVDGVEVHNAKTKP | |
| | REEQYNSTYRVVSVLTVLHQDWLNGKEYKCKVSNKALGAPIE | |
| | KTISKAKGQPREPQVYTLPPCRDELTKNQVSLWCLVKGFYPS | |
| | DIAVEWESNGQPENNYKTTPPVLDSDGSFFLYSKLTVDKSRW | |
| | QQGNVFSCSVMHEALHNHYTQKSLSLSPGGGGGSGGGGSGGG | |
| | GSAPASSSTKKTQLQLEHLLLDLQMILNGINNYKNPKLTRML | |
| | TAKFAMPKKATELKHLQCLEEELKPLEEVLNGAQSKNFHLRP | |
| | RDLISNINVIVLELKGSETTFMCEYADETATIVEFLNRWITF | |
| | AQSIISTLT | |
| PD-1 IL2v - | EVQLLESGGGLVQPGGSLRLSCAASGFSFSSYTMSWVRQAPG | 9 |
| HC without | KGLEWVATISGGGRDIYYPDSVKGRFTISRDNSKNTLYLQMN | |
| IL2v (Fc | SLRAEDTAVYYCVLLTGRVYFALDSWGQGTLVTVSSASTKGP | |
| hole, | SVFPLAPSSKSTSGGTAALGCLVKDYFPEPVTVSWNSGALTS | |
| LALAPG) | GVHTFPAVLQSSGLYSLSSVVTVPSSSLGTQTYICNVNHKPS | |
| | NTKVDKKVEPKSCDKTHTCPPCPAPEAAGGPSVFLFPPKPKD | |
| | TLMISRTPEVTCVVVDVSHEDPEVKFNWYVDGVEVHNAKTKP | |
| | REEQYNSTYRVVSVLTVLHQDWLNGKEYKCKVSNKALGAPIE | |
| | KTISKAKGQPREPQVCTLPPSRDELTKNQVSLSCAVKGFYPS | |
| | DIAVEWESNGQPENNYKTTPPVLDSDGSFFLVSKLTVDKSRW | |
| | QQGNVFSCSVMHEALHNHYTQKSLSLSP | |
| PD-1 IL2v - | DIVMTQSPDSLAVSLGERATINCKASESVDTSDNSFIHWYQQ | 10 |
| LC | KPGQSPKLLIYRSSTLESGVPDRFSGSGSGTDFTLTISSLQA | |
| | EDVAVYYCQQNYDVPWTFGQGTKVEIKRTVAAPSVFIFPPSD | |
| | EQLKSGTASVVCLLNNFYPREAKVQWKVDNALQSGNSQESVT | |
| | EQDSKDSTYSLSSTLTLSKADYEKHKVYACEVTHQGLSSPVT | |
| | KSFNRGEC | |
| Murine | EVQLQESGPGLVKPSQSLSLTCSVTGYSITSSYRWNWIRKFP | 11 |
| surrogate | GNRLEWMGYINSAGISNYNPSLKRRISITRDTSKNQFFLQVN | |
| PD-1 IL2v - | SVTTEDAATYYCARSDNMGTTPFTYWGQGTLVTVSSAKTTPP | |
| HC with | SVYPLAPGSAAQTNSMVTLGCLVKGYFPEPVTVTWNSGSLSS | |
| IL2v | GVHTFPAVLQSDLYTLSSSVTVPSSTWPSQTVTCNVAHPASS | |
| | TKVDKKIVPRDCGCKPCICTVPEVSSVFIFPPKPKDVLTITL | |
| | TPKVTCVVVAISKDDPEVQFSWFVDDVEVHTAQTKPREEQIN | |
| | STFRSVSELPIMHQDWLNGKEFKCRVNSAAFGAPIEKTISKT | |
| | KGRPKAPQVYTIPPPKEQMAKDKVSLTCMITNFFPEDITVEW | |
| | QWNGQPAENYDNTQPIMDTDGSYFVYSDLNVQKSNWEAGNTF | |
| | TCSVLHEGLHNHHTEKSLSHSPGGGGGSGGGGSGGGGSAPAS | |
| | SSTSSSTAEAQQQQQQQQQQQQHLEQLLMDLQELLSRMENYR | |
| | NLKLPRMLTAKFALPKQATELKDLQCLEDELGPLRHVLDGTQ | |
| | SKSFQLEDAENFISNIRVTVVKLKGSDNTFECQFDDESATVV | |
| | DFLRRWIAFAQSIISTSPQ | |
| Murine | EVQLQESGPGLVKPSQSLSLTCSVTGYSITSSYRWNWIRKFP | 12 |
| surrogate | GNRLEWMGYINSAGISNYNPSLKRRISITRDTSKNQFFLQVN | |
| PD-1 IL2v - | SVTTEDAATYYCARSDNMGTTPFTYWGQGTLVTVSSAKTTPP | |
| HC without | SVYPLAPGSAAQTNSMVTLGCLVKGYFPEPVTVTWNSGSLSS | |
| IL2v | GVHTFPAVLQSDLYTLSSSVTVPSSTWPSQTVTCNVAHPASS | |
| | TKVDKKIVPRDCGCKPCICTVPEVSSVFIFPPKPKDVLTITL | |
| | TPKVTCVVVAISKDDPEVQFSWFVDDVEVHTAQTKPREEQIN | |
| | STFRSVSELPIMHQDWLNGKEFKCRVNSAAFGAPIEKTISKT | |
| | KGRPKAPQVYTIPPPKKQMAKDKVSLTCMITNFFPEDITVEW | |
| | QWNGQPAENYKNTQPIMKTDGSYFVYSKLNVQKSNWEAGNTF | |
| | TCSVLHEGLHNHHTEKSLSHSPGK | |
| Murine | DIVMTQGTLPNPVPSGESVSITCRSSKSLLYSDGKTYLNWYL | 13 |
| surrogate | QRPGQSPQLLIYWMSTRASGVSDRFSGSGSGTDFTLKISGVE | |
| PD-1 IL2v - | AEDVGIYYCQQGLEFPTFGGGTKLELKRTDAAPTVSIFPPSS | |
| LC | EQLTSGGASVVCFLNNFYPKDINVKWKIDGSERQNGVLNSWT | |
| | DQDSKDSTYSMSSTLTLTKDEYERHNSYTCEATHKTSTSPIV | |
| | KSFNRNEC | |
| TYRP1 VH - | QVQLVQSGAEVKKPGASVKVSCKASGFNIKDYFLHWVRQAPG | 14 |
| CH1(EE) - | QGLEWMGWINPDNGNTVYAQKFQGRVTMTADTSTSTVYMELS | |
| CD3 VL - | SLRSEDTAVYYCTRRDYTYEKAALDYWGQGTLVTVSSASTKG | |
| CH1 - Fc | PSVFPLAPSSKSTSGGTAALGCLVEDYFPEPVTVSWNSGALT | |
| (knob, | SGVHTFPAVLQSSGLYSLSSVVTVPSSSLGTQTYICNVNHKP | |
| PGLALA) | SNTKVDEKVEPKSCDGGGGSGGGGSQAVVTQEPSLTVSPGGT | |
| | VTLTCGSSTGAVTTSNYANWVQEKPGQAFRGLIGGTNKRAPG | |
| | TPARFSGSLLGGKAALTLSGAQPEDEAEYYCALWYSNLWVFG | |
| | GGTKLTVLSSASTKGPSVFPLAPSSKSTSGGTAALGCLVKDY | |
| | FPEPVTVSWNSGALTSGVHTFPAVLQSSGLYSLSSVVTVPSS | |
| | SLGTQTYICNVNHKPSNTKVDKKVEPKSCDKTHTCPPCPAPE | |
| | AAGGPSVFLFPPKPKDTLMISRTPEVTCVVVDVSHEDPEVKF | |
| | NWYVDGVEVHNAKTKPREEQYNSTYRVVSVLTVLHQDWLNGK | |
| | EYKCKVSNKALGAPIEKTISKAKGQPREPQVYTLPPCRDELT | |
| | KNQVSLWCLVKGFYPSDIAVEWESNGQPENNYKTTPPVLDSD | |
| | GSFFLYSKLTVDKSRWQQGNVFSCSVMHEALHNHYTQKSLSL | |
| | SP | |
| TYRP1 VH - | QVQLVQSGAEVKKPGASVKVSCKASGFNIKDYFLHWVRQAPG | 15 |
| CH1(EE) - | QGLEWMGWINPDNGNTVYAQKFQGRVTMTADTSTSTVYMELS | |
| Fc (hole, | SLRSEDTAVYYCTRRDYTYEKAALDYWGQGTLVTVSSASTKG | |
| PGLALA) | PSVFPLAPSSKSTSGGTAALGCLVEDYFPEPVTVSWNSGALT | |
| | SGVHTFPAVLQSSGLYSLSSVVTVPSSSLGTQTYICNVNHKP | |
| | SNTKVDEKVEPKSCDKTHTCPPCPAPEAAGGPSVFLFPPKPK | |
| | DTLMISRTPEVTCVVVDVSHEDPEVKFNWYVDGVEVHNAKTK | |
| | PREEQYNSTYRVVSVLTVLHQDWLNGKEYKCKVSNKALGAPI | |
| | EKTISKAKGQPREPQVCTLPPSRDELTKNQVSLSCAVKGFYP | |
| | SDIAVEWESNGQPENNYKTTPPVLDSDGSFFLVSKLTVDKSR | |
| | WQQGNVFSCSVMHEALHNHYTQKSLSLSP | |
| TYRP1 VL - | DIQMTQSPSSLSASVGDRVTITCRASGNIYNYLAWYQQKPGK | 16 |
| CL(RK) | VPKLLIYDAKTLADGVPSRFSGSGSGTDFTLTISSLQPEDVA | |
| | TYYCQHFWSLPFTFGQGTKLEIKRTVAAPSVFIFPPSDRKLK | |
| | SGTASVVCLLNNFYPREAKVQWKVDNALQSGNSQESVTEQDS | |
| | KDSTYSLSSTLTLSKADYEKHKVYACEVTHQGLSSPVTKSFN | |
| | RGEC | |
| CD3 VH - | EVQLLESGGGLVQPGGSLRLSCAASGFQFSSYAMNWVRQAPG | 17 |
| CL | KGLEWVSRIRSKYNNYATYYADSVKGRFTISRDDSKNTLYLQ | |
| | MNSLRAEDTAVYYCVRHTTFPSSYVSYYGYWGQGTLVTVSSA | |
| | SVAAPSVFIFPPSDEQLKSGTASVVCLLNNFYPREAKVQWKV | |
| | DNALQSGNSQESVTEQDSKDSTYSLSSTLTLSKADYEKHKVY | |
| | ACEVTHQGLSSPVTKSFNRGEC | |
| murine | EVQLVESGGGLVQPGKSLKLSCEASGFTFSGYGMHWVRQAPG | 18 |
| surrogate | RGLESVAYITSSSINIKYADAVKGRFTVSRDNAKNLLFLQMN | |
| CD3 VH - | ILKSEDTAMYYCARFDWDKNYWGQGTMVTVSSASDAAPTVSI | |
| CL - TYRP1 | FPPSSEQLTSGGASVVCFLNNFYPKDINVKWKIDGSERQNGV | |
| VH - CH1 - | LNSWTDQDSKDSTYSMSSTLTLTKDEYERHNSYTCEATHKTS | |
| Fc (DAPG, | TSPIVKSFNRNECGGGGSGGGGSEVQLQQSGAELVRPGALVK | |
| DD) | LSCKTSGFNIKDYFLHWVRQRPDQGLEWIGWINPDNGNTVYD | |
| | PKFQGTASLTADTSSNTVYLQLSGLTSEDTAVYFCTRRDYTY | |
| | EKAALDYWGQGASVIVFSAAKTTPPSVYPLAPGSAAQTNSMV | |
| | TLGCLVKGYFPEPVTVTWNSGSLSSGVHTFPAVLQSDLYTLS | |
| | SSVTVPSSTWPSQTVTCNVAHPASSTKVDKKIVPRDCGCKPC | |
| | ICTVPEVSSVFIFPPKPKDVLTITLTPKVTCVVVAISKDDPE | |
| | VQFSWFVDDVEVHTAQTKPREEQINSTFRSVSELPIMHQDWL | |
| | NGKEFKCRVNSAAFGAPIEKTISKTKGRPKAPQVYTIPPPKE | |
| | QMAKDKVSLTCMITNFFPEDITVEWQWNGQPAENYDNTQPIM | |
| | DTDGSYFVYSDLNVQKSNWEAGNTFTCSVLHEGLHNHHTEKS | |
| | LSHSPGK | |
| murine | EVQLQQSGAELVRPGALVKLSCKTSGFNIKDYFLHWVRQRPD | 19 |
| surrogate | QGLEWIGWINPDNGNTVYDPKFQGTASLTADTSSNTVYLQLS | |
| TYRP1 VH - | GLTSEDTAVYFCTRRDYTYEKAALDYWGQGASVIVFSAAKTT | |
| CH1 - | PPSVYPLAPGSAAQTNSMVTLGCLVKGYFPEPVTVTWNSGSL | |
| Fc | SSGVHTFPAVLQSDLYTLSSSVTVPSSTWPSQTVTCNVAHPA | |
| (DAPG, KK) | SSTKVDKKIVPRDCGCKPCICTVPEVSSVFIFPPKPKDVLTI | |
| | TLTPKVTCVVVAISKDDPEVQFSWFVDDVEVHTAQTKPREEQ | |
| | INSTFRSVSELPIMHQDWLNGKEFKCRVNSAAFGAPIEKTIS | |
| | KTKGRPKAPQVYTIPPPKKQMAKDKVSLTCMITNFFPEDITV | |
| | EWQWNGQPAENYKNTQPIMKTDGSYFVYSKLNVQKSNWEAGN | |
| | TFTCSVLHEGLHNHHTEKSLSHSPGK | |
| murine | AIQMSQSPASLSASVGETVTITCRASGNIYNYLAWYQQKQGK | 20 |
| surrogate | SPHLLVYDAKTLADGVPSRFSGSGSGTQYSLKISSLQTEDSG | |
| TYRP1 VL - | NYYCQHFWSLPFTFGSGTKLEIKRADAAPTVSIFPPSSEQLT | |
| CL | SGGASVVCFLNNFYPKDINVKWKIDGSERQNGVLNSWTDQDS | |
| | KDSTYSMSSTLTLTKDEYERHNSYTCEATHKTSTSPIVKSFN | |
| | RNEC | |
| murine | DIQMTQSPSSLPASLGDRVTINCQASQDISNYLNWYQQKPGK | 21 |
| surrogate | APKLLIYYTNKLADGVPSRFSGSGSGRDSSFTISSLESEDIG | |
| CD3 VL - | SYYCQQYYNYPWTFGPGTKLEIKSSAKTTPPSVYPLAPGSAA | |
| CH1 | QTNSMVTLGCLVKGYFPEPVTVTWNSGSLSSGVHTFPAVLQS | |
| | DLYTLSSSVTVPSSTWPSQTVTCNVAHPASSTKVDKKIVPRD | |
| | C | |
| Wild-type | APTSSSTKKTQLQLEHLLLDLQMILNGINNYKNPKLTRMLTF | 22 |
| human IL-2 | KFYMPKKATELKHLQCLEEELKPLEEVLNLAQSKNFHLRPRD | |
| | LISNINVIVLELKGSETTFMCEYADETATIVEFLNRWITFAQ | |
| | SIISTLT | |
| human | QFPRQCATVEALRSGMCCPDLSPVSGPGTDRCGSSSGRGRCE | 23 |
| TYRP1 | AVTADSRPHSPQYPHDGRDDREVWPLRFFNRTCHCNGNFSGH | |
| | NCGTCRPGWRGAACDQRVLIVRRNLLDLSKEEKNHFVRALDM | |
| | AKRTTHPLFVIATRRSEEILGPDGNTPQFENISIYNYFVWTH | |
| | YYSVKKTFLGVGQESFGEVDFSHEGPAFLTWHRYHLLRLEKD | |
| | MQEMLQEPSFSLPYWNFATGKNVCDICTDDLMGSRSNFDSTL | |
| | ISPNSVFSQWRVVCDSLEDYDTLGTLCNSTEDGPIRRNPAGN | |
| | VARPMVQRLPEPQDVAQCLEVGLFDTPPFYSNSTNSFRNTVE | |
| | GYSDPTGKYDPAVRSLHNLAHLFLNGTGGQTHLSPNDPIFVL | |
| | LHTFTDAVFDEWLRRYNADISTFPLENAPIGHNRQYNMVPFW | |
| | PPVTNTEMFVTAPDNLGYTYEIQWPSREFSVPEIIAIAVVGA | |
| | LLLVALIFGTASYLIRARRSMDEANQPLLTDQYQCYAEEYEK | |
| | LQNPNQSVV | |
| muFAP HC- | EVQLLESGGGLVQPGGSLRLSCAASGFTFSSHAMSWVRQAPG | 24 |
| Fc (DD)- | KGLEWVSAIWASGEQYYADSVKGRFTISRDNSKNTLYLQMNS | |
| muIL2v | LRAEDTAVYYCAKGWLGNFDYWGQGTLVTVSSAKTTPPSVYP | |
| | LAPGSAAQTNSMVTLGCLVKGYFPEPVTVTWNSGSLSSGVHT | |
| | FPAVLQSDLYTLSSSVTVPSSTWPSETVTCNVAHPASSTKVD | |
| | KKIVPRDCGCKPCICTVPEVSSVFIFPPKPKDVLTITLTPKV | |
| | TCVVVDISKDAPEVQFSWFVDDVEVHTAQTQPREEQFNSTFR | |
| | SVSELPIMHQDWLNGKEFKCRVNSAAFGAPIEKTISKTKGRP | |
| | KAPQVYTIPPCKEQMAKDKVSLWCMITDFFPEDITVEWQWNG | |
| | QPAENYKNTQPIMDTDGSYFVYSKLNVQKSNWEAGNTFTCSV | |
| | LHEGLHNHHTEKSLSHSPGGGGGSGGGGSGGGGSAPASSSTS | |
| | SSTAEAQQQQQQQQQQQQHLEQLLMDLQELLSRMENYRNLKL | |
| | PRMLTAKFALPKQATELKDLQCLEDELGPLRHVLDGTQSKSF | |
| | QLEDAENFISNIRVTVVKLKGSDNTFECQFDDESATVVDFLR | |
| | RWIAFAQSIISTSPQ | |
| muFAP HC- | EVQLLESGGGLVQPGGSLRLSCAASGFTFSSHAMSWVRQAPG | 25 |
| Fc (KK) | KGLEWVSAIWASGEQYYADSVKGRFTISRDNSKNTLYLQMNS | |
| | LRAEDTAVYYCAKGWLGNFDYWGQGTLVTVSSAKTTPPSVYP | |
| | LAPGSAAQTNSMVTLGCLVKGYFPEPVTVTWNSGSLSSGVHT | |
| | FPAVLQSDLYTLSSSVTVPSSTWPSETVTCNVAHPASSTKVD | |
| | KKIVPRDCGCKPCICTVPEVSSVFIFPPKPKDVLTITLTPKV | |
| | TCVVVDISKDAPEVQFSWFVDDVEVHTAQTQPREEQFNSTFR | |
| | SVSELPIMHQDWLNGKEFKCRVNSAAFGAPIEKTISKTKGRP | |
| | KAPQVCTIPPPKEQMAKDKVSLSCAITDFFPEDITVEWQWNG | |
| | QPAENYKNTQPIMDTDGSYFVVSKLNVQKSNWEAGNTFTCSV | |
| | LHEGLHNHHTEKSLSHSPGK | |
| muFAPLC | EIVLTQSPGTLSLSPGERATLSCRASQSVSRSYLAWYQQKPG | 26 |
| | QAPRLLIIGASTRATGIPDRFSGSGSGTDFTLTISRLEPEDF | |
| | AVYYCQQGQVIPPTFGQGTKVEIKRADAAPTVSIFPPSSEQL | |
| | TSGGASVVCFLNNFYPKDINVKWKIDGSERQNGVLNSWTDQD | |
| | SKDSTYSMSSTLTLTKDEYERHNSYTCEATHKTSTSPIVKSF | |
| | NRNEC | |
| muPD1 HC | EVQLQESGPGLVKPSQSLSLTCSVTGYSITSSYRWNWIRKFP | 27 |
| | GNRLEWMGYINSAGISNYNPSLKRRISITRDTSKNQFFLQVN | |
| | SVTTEDAATYYCARSDNMGTTPFTYWGQGTLVTVSSASTTAP | |
| | SVYPLAPVCGDTTGSSVTLGCLVKGYFPEPVTLTWNSGSLSS | |
| | GVHTFPAVLQSDLYTLSSSVTVTSSTWPSQSITCNVAHPASS | |
| | TKVDKKIEPRGPTIKPCPPCKCPAPNAAGGPSVFIFPPKIKD | |
| | VLMISLSPIVTCVVVDVSEDDPDVQISWFVNNVEVHTAQTQT | |
| | HREDYNSTLRVVSALPIQHQDWMSGKEFKCKVNNKDLGAPIE | |
| | RTISKPKGSVRAPQVYVLPPPEEEMTKKQVTLTCMVTDFMPE | |
| | DIYVEWTNNGKTELNYKNTEPVLDSDGSYFMYSKLRVEKKNW | |
| | VERNSYSCSVVHEGLHNHHTTKSFSRTPGK | |
| muPD1 LC | DIVMTQGTLPNPVPSGESVSITCRSSKSLLYSDGKTYLNWYL | 28 |
| | QRPGQSPQLLIYWMSTRASGVSDRFSGSGSGTDFTLKISGVE | |
| | AEDVGIYYCQQGLEFPTFGGGTKLELKRTDAAPTVSIFPPSS | |
| | EQLTSGGASVVCFLNNFYPKDINVKWKIDGSERQNGVLNSWT | |
| | DQDSKDSTYSMSSTLTLTKDEYERHNSYTCEATHKTSTSPIV | |
| | KSFNRNEC | |

1. A PD-1-targeted IL-2 variant immunoconjugate in combination with an anti-TYRP1/anti-CD3 bispecific antibody for use as a combination therapy in the treatment of cancer, for use as a combination therapy in the prevention or treatment of metastasis, for use as a combination therapy in treating or delaying progression of an immune related disease such as tumor immunity, or for use as a combination therapy in stimulating an immune response or function, such as T cell activity,
wherein the PD-1-targeted IL-2 variant immunoconjugate comprises a heavy chain variable domain VH of SEQ ID NO: 1 and a light chain variable domain VL of SEQ ID NO: 2, and the polypeptide sequence of SEQ ID NO: 3,
and wherein the anti-TYRP1/anti-CD3 bispecific antibody comprises a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7.

2. The PD-1-targeted IL-2 variant immunoconjugate in combination with an anti-TYRP1/anti-CD3 bispecific antibody according to claim 1, for use in the treatment of breast cancer, lung cancer, colon cancer, ovarian cancer, melanoma cancer, bladder cancer, renal cancer, kidney cancer, liver cancer, head and neck cancer, colorectal cancer, melanoma, pancreatic cancer, gastric carcinoma cancer, esophageal cancer, mesothelioma, prostate cancer, leukemia, lymphomas, or myelomas.

3. The PD-1-targeted IL-2 variant immunoconjugate in combination with an anti-TYRP1/anti-CD3 bispecific antibody according to claim 1, characterized in that the antibody component of the immunoconjugate and the bispecific antibody are of human IgG₁ or human IgG₄ subclass.

4. The PD-1-targeted IL-2 variant immunoconjugate in combination with an anti-TYRP1/anti-CD3 bispecific antibody according to claim 1, characterized in that the antibody component of the immunoconjugate and the bispecific antibody have reduced or minimal effector function.

5. The PD-1-targeted IL-2 variant immunoconjugate in combination with an anti-TYRP1/anti-CD3 bispecific antibody according to claim 4, wherein the minimal effector function results from an effectorless Fc mutation.

6. The PD-1-targeted IL-2 variant immunoconjugate in combination with an anti-TYRP1/anti-CD3 bispecific antibody according to claim 5, wherein the effectorless Fc mutation is L234A/L235A or L234A/L235A/P329G or N297A or D265A/N297A.

7. The PD-1-targeted IL-2 variant immunoconjugate in combination with an anti-TYRP1/anti-CD3 bispecific antibody according to claim 1, wherein the PD-1-targeted IL-2 variant immunoconjugate comprises:
i) a polypeptide sequence of SEQ ID NO: 8 or SEQ ID NO: 9 or SEQ ID NO: 10,
ii) the polypeptide sequence of SEQ ID NO: 8, and SEQ ID NO: 9 and SEQ ID NO: 10, or
iii) the polypeptide sequence of SEQ ID NO: 11 and SEQ ID NO: 12 and SEQ ID NO: 13
and wherein the anti-TYRP1/anti-CD3 bispecific antibody comprises a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7.

8. A PD-1-targeted IL-2 variant immunoconjugate in combination with an anti-TYRP1/anti-CD3 bispecific antibody according to claim 1, wherein the PD-1-targeted IL-2 variant immunoconjugate comprises:
i) a polypeptide sequence of SEQ ID NO: 8 or SEQ ID NO: 9 or SEQ ID NO: 10,
ii) the polypeptide sequence of SEQ ID NO: 8, and SEQ ID NO: 9 and SEQ ID NO: 10, or
iii) the polypeptide sequence of SEQ ID NO: 11 and SEQ ID NO: 12 and SEQ ID NO: 13,
and wherein the anti-TYRP1/anti-CD3 bispecific antibody comprises:
i) a polypeptide sequence of SEQ ID NO: 14 or SEQ ID NO: 15 or SEQ ID NO: 16 or SEQ ID NO: 17,
ii) a polypeptide sequence of SEQ ID NO: 14 and SEQ ID NO: 15 and SEQ ID NO: 16 and SEQ ID NO: 17, or
iii) a polypeptide sequence of SEQ ID NO: 18 and SEQ ID NO: 19 and SEQ ID NO: 20 and SEQ ID NO: 21.

9. A PD-1-targeted IL-2 variant immunoconjugate in combination with an anti-TYRP1/anti-CD3 bispecific antibody for use in:
i) Inhibition of tumor growth in a tumor; and/or
ii) Enhancing median and/or overall survival of subjects with a tumor;
wherein PD-1 is presented on immune cells, particularly T cells, or in a tumor cell environment,
wherein the PD-1 targeted IL-2 variant immunoconjugate used in the combination therapy is characterized in comprising:
i) a heavy chain variable domain VH of SEQ ID NO: 1 and a light chain variable domain VL of SEQ ID NO: 2, and the polypeptide sequence of SEQ ID NO: 3,
ii) a polypeptide sequence of SEQ ID NO: 8 or SEQ ID NO: 9 or SEQ ID NO: 10,
iii) the polypeptide sequence of SEQ ID NO: 8, and SEQ ID NO: 9 and SEQ ID NO: 10, or
iv) the polypeptide sequence of SEQ ID NO: 11 and SEQ ID NO: 12 and SEQ ID NO: 13,
and the anti-TYRP1/anti-CD3 bispecific antibody used in the combination therapy is characterized in comprising:
i) a first antigen binding moiety comprising a heavy chain variable domain VH of SEQ ID NO: 4 and a light chain variable domain VL of SEQ ID NO: 5 and second antigen binding moiety comprising a heavy chain variable domain VH of SEQ ID NO: 6 and a light chain variable domain VL of SEQ ID NO: 7;
ii) a polypeptide sequence of SEQ ID NO: 14 or SEQ ID NO: 15 or SEQ ID NO: 16 or SEQ ID NO: 17,
iii) a polypeptide sequence of SEQ ID NO: 14 and SEQ ID NO: 15 and SEQ ID NO: 16 and SEQ ID NO: 17, or
iv) a polypeptide sequence of SEQ ID NO: 18 and SEQ ID NO: 19 and SEQ ID NO: 20 and SEQ ID NO: 21.

10. A PD-1-targeted IL-2 variant immunoconjugate in combination with an anti-TYRP1/anti-CD3 bispecific antibody according to claim 1, wherein the PD-1 targeted IL-2 variant immunoconjugate used in the combination therapy is characterized in comprising the polypeptide sequences of SEQ ID NO: 1, SEQ ID NO: 2, and SEQ ID NO: 3, and wherein the anti-TYRP1/anti-CD3 bispecific antibody used in the combination therapy is characterized in comprising a polypeptide sequence of SEQ ID NO: 14 and SEQ ID NO: 15 and SEQ ID NO: 16 and SEQ ID NO: 17.

11. The PD-1-targeted IL-2 variant immunoconjugate in combination with an anti-TYRP1/anti-CD3 bispecific antibody according to claim 1, wherein the patient is treated with or was pre-treated with immunotherapy.

12. A PD-1-targeted IL-2 variant immunoconjugate in combination with an anti-TYRP1/anti-CD3 bispecific antibody according to claim 9, wherein the PD-1 targeted IL-2 variant immunoconjugate used in the combination therapy is characterized in comprising the polypeptide sequences of SEQ ID NO: 1, SEQ ID NO: 2, and SEQ ID NO: 3, and wherein the anti-TYRP1/anti-CD3 bispecific antibody used in the combination therapy is characterized in comprising a polypeptide sequence of SEQ ID NO: 14 and SEQ ID NO: 15 and SEQ ID NO: 16 and SEQ ID NO: 17.

13. The PD-1-targeted IL-2 variant immunoconjugate in combination with an anti-TYRP1/anti-CD3 bispecific antibody according to claim 9, wherein the patient is treated with or was pre-treated with immunotherapy.
